(19) United States
(12) Patent Application Publication
(10) Pub. No.: US 2002/0192750 A1
(43) Pub. Date: Dec. 19, 2002

(54) Neuropilin homolog zcub5

(76) Inventors: Brian A. Fox, Seattle, WA (US); Zeren Gao, Redmond, WA (US); Kimberly E. Shoemaker, Bellevue, WA (US)

Correspondence Address: Gary E. Parker, ZymoGenetics, Inc., 1201 Eastlake Avenue East, Seattle, WA, 98102, US

(21) Appl. No.: 10/003,132

(22) Filed: Nov. 15, 2001

Related U.S. Application Data
(60) Provisional application No. 60/249,004, filed on Nov. 15, 2000.

(51) Int. Cl. C07K 16/46; C12P 21/02; C07H 21/04; C12N 5/06

(52) U.S. Cl. 435/69.1; 435/326; 435/320.1; 530/391.1; 536/23.53

(57) ABSTRACT

Novel polypeptides, materials and methods for making them, and method of use are disclosed. The polypeptides comprise residues 41-150 of SEQ ID NO:2 or residues 32-141 of SEQ ID NO:4, and may be prepared as polypeptide fusions comprise heterologous sequences, such as affinity tags. The polypeptides and polynucleotides encoding them may be used within a variety of therepeutic, diagnostic, and research applications.

### CROSS-REFERENCE TO RELATED APPLICATION

This application claims the benefit of provisional application No. 60/249,004, filed Nov. 15, 2000.

### BACKGROUND OF THE INVENTION

In multicellular animals, cell growth, differentiation, and migration are controlled by polypeptide growth factors. Polypeptide growth factors influence cellular events by binding to cell-surface receptors, many of which are tyrosine kinases. Binding initiates a chain of signalling events within the cell, which ultimately results in phenotypic changes, such as cell division, protease production, and cell migration. These growth factors play a role in both normal development and pathogenesis, including the development of solid tumors.

Growth factors can be classified into families on the basis of structural similarities. One such family, the PDGF (platelet derived growth factor) family, is characterized by a dimeric structure stabilized by disulfide bonds. This family includes PDGF, the placental growth factors (PlGFs), and the vascular endothelial growth factors (VEGFs). Four vascular endothelial growth factors have been identified: VEGF, also known as vascular permeability factor (Dvorak et al., Am. J. Pathol. 146:1029-1039, 1995); VEGF-B (Olofsson et al., Proc. Natl. Acad. Sci. USA 93:2567-2581, 1996; Hayward et al., WIPO Publication WO 96/27007); VEGF-C (Joukov et al., EMBO J. 15:290-298, 1996); and VEGF-D (Oliviero, WO 97/12972; Achen et al., WO 98/07832). Five VEGF polypeptides (121, 145, 165, 189, and 206 amino acids) arise from alternative splicing of the VEGF MRNA.

VEGFs stimulate the development of vasculature through a process known as angiogenesis, wherein vascular endothelial cells re-enter the cell cycle, degrade underlying basement membrane, and migrate to form new capillary sprouts. These cells then differentiate, and mature vessels are formed. This process of growth and differentiation is regulated by a balance of pro-angiogenic and anti-angiogenic factors. Angiogenesis is central to normal formation and repair of tissue, occuring in embryo development and wound healing. Angiogenesis is also a factor in the development of certain diseases, including solid tumors, rheumatoid arthritis, diabetic retinopathy, macular degeneration, and atherosclerosis.

Three receptors for VEGF have been identified: KDR/Flk-1 (Matthews et al., Proc. Natl. Acad. Sci. USA 88:9026-9030, 1991), Flt-1 (de Vries et al., Science 255:989-991, 1992), and neuropilin-1 (Soker et al., Cell 92:735-745, 1998).

Neuropilin-1 is a cell-surface glycoprotein that was initially identified in Xenopus tadpole nervous tissues, then in chicken, mouse, and human. The primary structure of neuropilin-1 is highly conserved among these vertebrate species. Neuropilin-1 has also been demonstrated to be a receptor for various members of the semaphorin family, including semaphorin III (Kolodkin et al., Cell 90:753-762, 1997), Sema E and Sema IV (Chen et al., Neuron 19:547-559, 1997). A variety of activities have been associated with the binding of neuropilin-1 to its ligands. For example, binding of semaphorin III to neuropilin-1 can induce neuronal growth cone collapse and repulsion of neurites in vitro (Kitsukawa et al., Neuron 19: 995-1005, 1997). NP-1 may thus play in role in development and/or maintenance of both the vasculature and the nervous system.

In mice, neuropilin-1 is expressed in the cardiovascular system, nervous system, and limbs at particular developmental stages. Chimeric mice over-expressing neuropilin-1 were found to be embryonic lethal (Kitsukawa et al., Development 121:4309-4318, 1995). The chimeric embryos exhibited several morphological abnormalities, including excess capillaries and blood vessels, dilation of blood vessels, malformed hearts, ectopic sprouting and defasciculation of nerve fibers, and extra digits. All of these abnormalities occurred in the organs in which neuropilin-1 is expressed in normal development. Mice lacking the neuropilin-1 gene have severe cardiovascular abnormalities, including impairment of vascular network formation in the central and peripheral nervous systems (Takashima et al., American Heart Association 1998 Meeting, Abstract # 3178).

Neuropilin-1 (NP-1) displays selective binding activity for VEGF₁₆₅ over VEGF₁₂₁. It has been shown to be expressed on vascular endothelial cells and tumor cells in vitro. When NP-1 is co-expressed in cells with KDR, NP-1 enhances the binding of VEGF₁₆₅ to KDR and VEGF₁₆₅-mediated chemotaxis. Conversely, inhibition of VEGF₁₆₅ binding to NP-1 inhibits its binding to KDR and its mitogenic activity for endothelial cells (Soker, et al., ibid.). NP-1 is also a receptor for PlGF-2 (Migdal et al., J. Biol. Chem. 273: 22272-22278, 1998) and binds to placenta growth factor (PlGF), various semaphorins (which inhibit growth of axons), and the VEGF receptor Flt-1 (Fuh et al., J. Biol. Chem. 275:26690-26695, 2000).

NP-1 contains a CUB domain, an extracellular domain of about 100 to 120 amino acid residues characterized by a conserved sequence motif and a predicted beta barrel structure. This domain is believed to mediate the binding of NP-1 to VEGF. CUB domains are found in a number of other, functionally diverse, mostly developmentally regulated proteins, including growth factors, cell surface receptors, and membrane and extracellular matrix-bound proteases. CUB domains occur in mammalian complement subcomponents C1s and C1r, human bone morphogenetic protein-1, zvegf3/PDGF-C (WO 00/18219 and WO 00/34474) and zvegf4/PDGF-D (WO 00/27879 and WO 00/34474), porcine seminal plasma protein and bovine acidic seminal fluid protein, hamster serine protease Casp, mammalian complement-activating component of Ra-reactive factor (RARF, also known as P100), vertebrate enteropeptidase (EC 3.4.21.9), vertebrate bone morphogenic protein 1 (BMP-1), sea urchin blastula proteins BP10 and SpAN, fibropellins I and III from sea urchin, mammalian hyaluronate-binding protein TSG-6 (or PS4), mammalian spermadhesins, Xenopus embryonic protein UVS2, and X. laevis tolloid-like protein. See, Takagi et al., Neuron 7:295-307, 1991; Soker et al., ibid.; Wozney et al., Science 242:1528-1534, 1988; Romero et al., Nat. Struct. Biol. 4:783-788, 1997; Lin et al., Dev. Growth Differ. 39:43-51, 1997; Bork and Beckmann, J. Mol. Biol. 231:539-545, 1993; and Bork, FEBS Lett. 282:9-12, 1991.

The role of growth factors, other regulatory molecules, and their receptors in controlling cellular processes makes them likely candidates and targets for therapeutic intervention. Platelet-derived growth factor, for example, has been disclosed for the treatment of periodontal disease (U.S. Pat. No. 5,124,316), gastrointestinal ulcers (U.S. Pat. No. 5,234,908), and dermal ulcers (Robson et al., Lancet 339:23-25, 1992). Inhibition of PDGF receptor activity has been shown to reduce intimal hyperplasia in injured baboon arteries (Giese et al., Restenosis Summit Vm, Poster Session #23, 1996; U.S. Pat. No. 5,620,687). VEGF has been shown to promote the growth of blood vessels in ischemic limbs (Isner et al., The Lancet 348:370-374, 1996), and members of this family have been proposed for use as wound-healing agents, for treatment of periodontal disease, for promoting endothelialization in vascular graft surgery, and for promoting collateral circulation following myocardial infarction (WIPO Publication No. WO 95/24473; U.S. Pat. No. 5,219,739). VEGFs are also useful for promoting the growth of vascular endothelial cells in culture. A soluble VEGF receptor (soluble flt-1) has been found to block binding of VEGF to cell-surface receptors and to inhibit the growth of vascular tissue in vitro (Biotechnology News 16(17):5-6, 1996).

### DESCRIPTION OF THE INVENTION

Within one aspect of the present invention there is provided an isolated polypeptide comprising at least fifteen contiguous amino acid residues of SEQ ID NO:2, SEQ ID NO:4, or SEQ ID NO:6. Within one embodiment of the invention the polypeptide is from 15 to 1800 amino acid residues in length. Within other embodiments, the polypeptide comprises residues 41-150 of SEQ ID NO:2 or residues 32-141 of SEQ ID NO:4. Within additional embodiments the polypeptide is operably linked to a second polypeptide selected from the group consisting of maltose binding protein, an immunoglobulin constant region, a polyhistidine tag, a peptide as shown in SEQ ID NO:7, and a peptide linker consisting of up to 25 amino acid residues. Within other embodiments the polypeptide comprises residues residues 41-412 of SEQ ID NO:2, residues 41-452 of SEQ ID NO:2, residues 35-150 of SEQ ID NO:2, residues 35-412 of SEQ ID NO:2, residues 35-452 of SEQ ID NO:2, residues 32-244 of SEQ ID NO:4, residues 26-141 of SEQ ID NO:4, or residues 26-244 of SEQ ID NO:4. Within other embodiments, the polypeptide further comprises an immunoglobulin constant region domain and hinge region.

Within a second aspect of the invention there is provided a dimerized polypeptide fusion comprising two polypeptide chains. Within one embodiment, each of the polypeptide chains comprises residues 41 to 150 of SEQ ID NO:2 joined to an IgG constant region domain and hinge region. Within a second embodiment, each of the polypeptide chains comprises residues 41 to 412 of SEQ ID NO:2 joined to an IgG constant region domain and hinge region. Within a third embodiment, each of the polypeptide chains comprises residues 32 to 141 of SEQ ID NO:4 joined to an IgG constant region domain and hinge region.

Within a third aspect of the invention there is provided an expression vector comprising the following operably linked elements: (a) a transcription promoter; (b) a DNA segment encoding a polypeptide comprising a sequence of amino acid residues selected from the group consisting of residues 41-150 of SEQ ID NO:2, residues 41-412 of SEQ ID NO:2, residues 41-452 of SEQ ID NO:2, residues 35-150 of SEQ ID NO:2, residues 35-412 of SEQ ID NO:2, residues 35-452 of SEQ ID NO:2, residues 32-141 of SEQ ID NO:4, residues 32-244 of SEQ ID NO:4, residues 26-141 of SEQ ID NO:4; and residues 26-244 of SEQ ID NO:4; and (c) a transcription terminator.

Within one embodiment the expression vector further comprises a secretory signal sequence operably linked to the DNA segment. Within a related embodiment the secretory signal sequence encodes residues 1-34 of SEQ ID NO:2 or residues 1-25 of SEQ ID NO:4. Within further embodiments the encoded polypeptide further comprises a maltose binding protein, an immunoglobulin constant region, a polyhistidine tag, a peptide as shown in SEQ ID NO:7, or a peptide linker consisting of up to 25 amino acid residues. Within a related embodiment, the encoded polypeptide further comprises an immunoglobulin constant region domain and hinge region.

Within a fourth aspect of the invention there is provided a cultured cell into which has been introduced an expression vector as disclosed above, wherein the cell expresses the DNA segment.

Within a fifth aspect of the invention there is provided a method of making a protein comprising the steps of culturing a cell as disclosed above under conditions whereby the DNA segment is expressed and the polypeptide is produced, and recovering the polypeptide. Within one embodiment the expression vector comprises a secretory signal sequence operably linked to the DNA segment, and the polypeptide is secreted by the cell and recovered from a medium in which the cell is cultured.

Within a sixth aspect of the invention there is provided a polypeptide produced by the method disclosed above.

Within a seventh aspect of the invention there is provided an antibody that specifically binds to a polypeptide as disclosed above. Within one embodiment the antibody is labeled to produce a detectable signal.

Within an eighth aspect of the invention there is provided a method of detecting, in a test sample, a polypeptide selected from the group consisting of (a) a polypeptide as shown in SEQ ID NO:2, and (b) proteolytic fragments of (a), the method comprising combining the test sample with an antibody as disclosed above under conditions whereby the antibody binds to the polypeptide, and detecting the presence of antibody bound to the polypeptide.

Within a ninth aspect of the invention there is provided a method of detecting, in a test sample, the presence of an antagonist of zcub5 activity, comprising the steps of (a) culturing a cell that is responsive to zcub5; (b) exposing the cell to a zcub5 polypeptide in the presence and absence of a test sample; (c) comparing levels of response to the zcub5 polypeptide, in the presence and absence of the test sample, by a biological or biochemical assay; and (d) determining from the comparison the presence of an antagonist of zcub5 activity in the test sample.

These and other aspects of the invention will become evident upon reference to the following detailed description of the invention and the attached drawings.

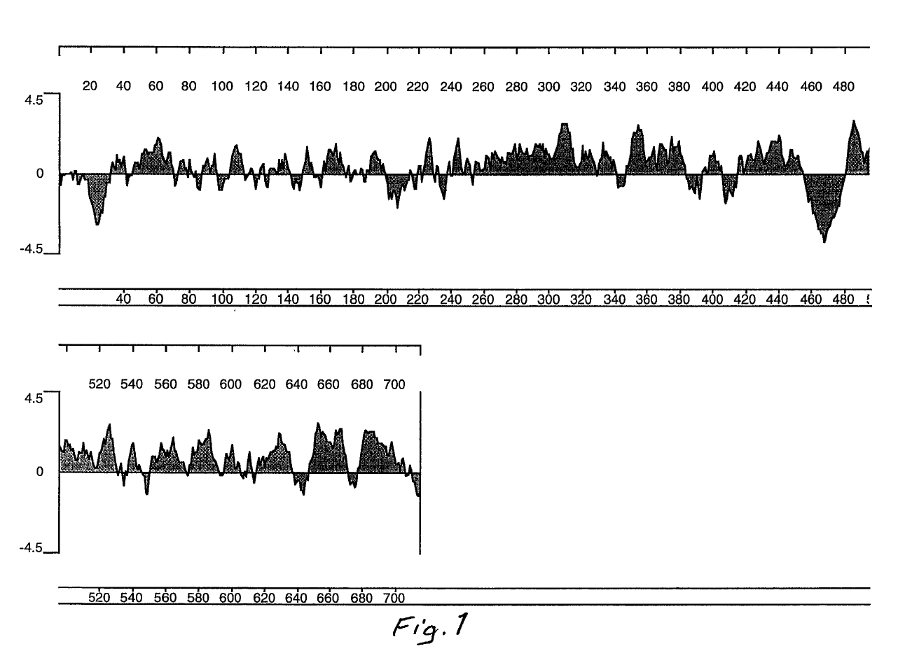

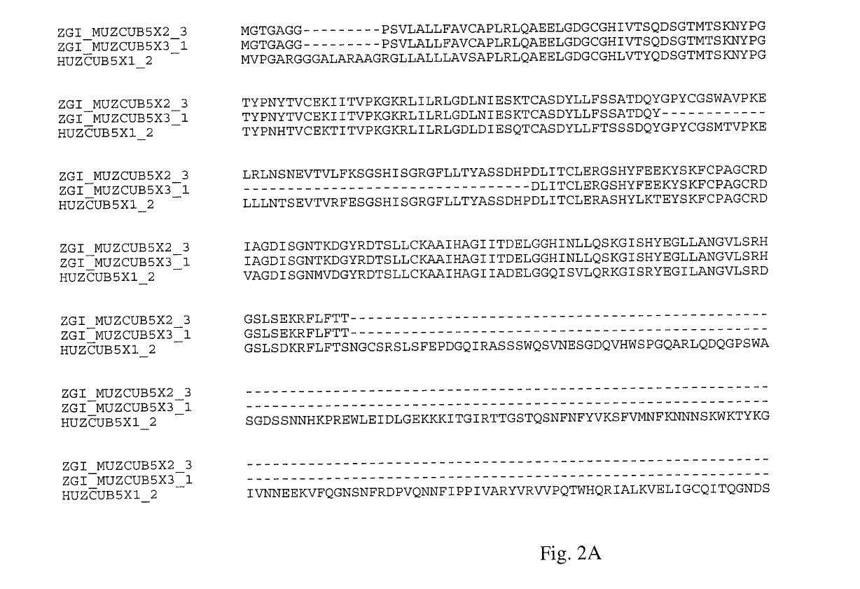

FIG. 1 is a Kyte-Doolittle hydrophilicity profile of the amino acid sequence shown in SEQ ID NO:2. The profile was prepared using Protean™ 3.14 (DNAStar, Madison, Wis.).

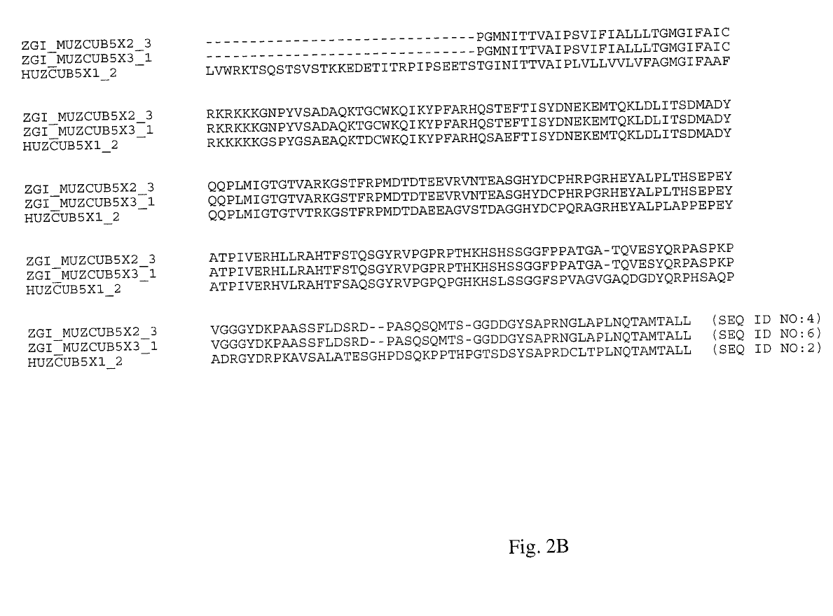

FIGS. 2A-2B are an alignment of SEQ ID NO:2, SEQ ID NO:4, and SEQ ID NO:6. Gaps have been introduced into the sequences to optimize the alignment in accordance with conventional methods.

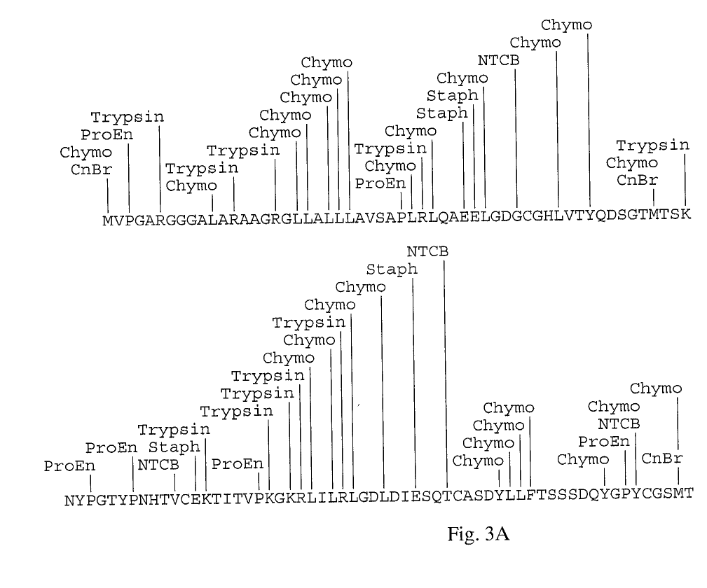

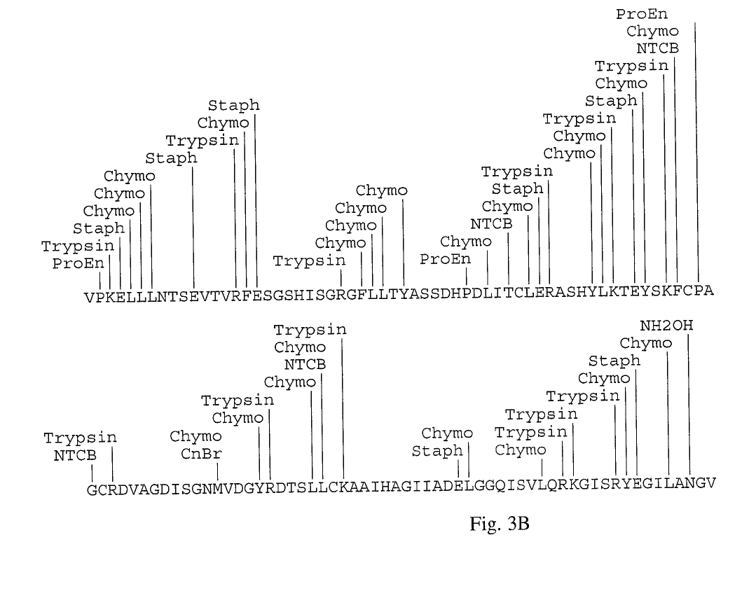

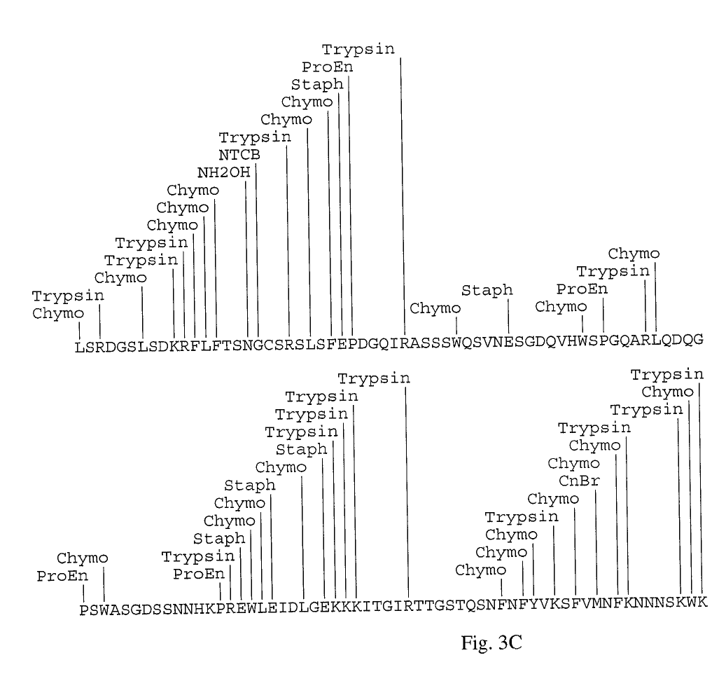

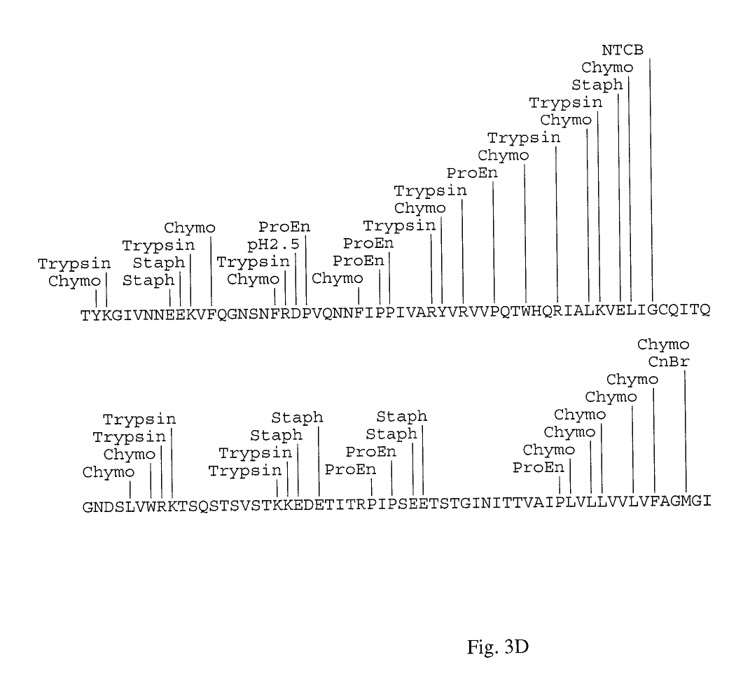

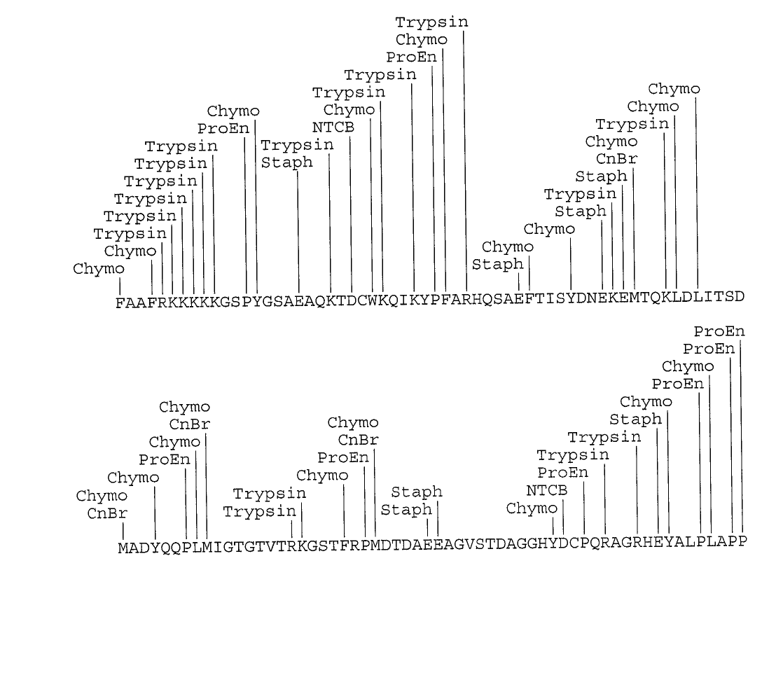

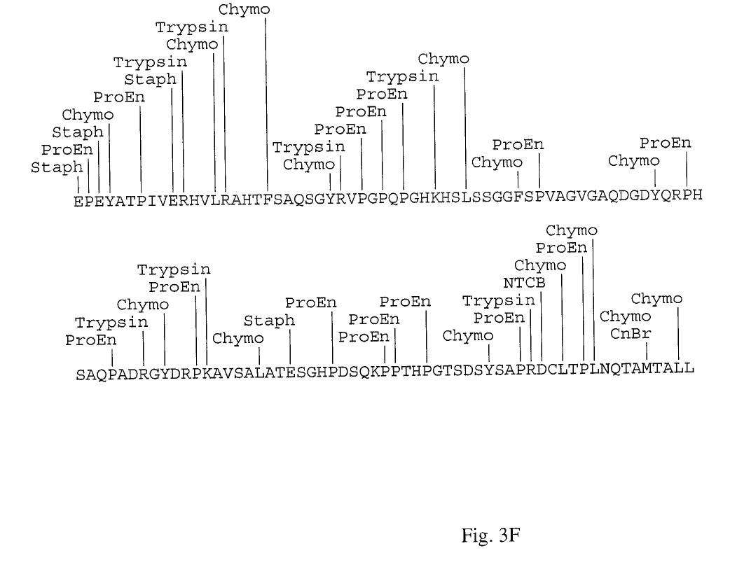

FIGS. 3A-3F are a partial proteolytic cleavage map of the polypeptide of SEQ ID NO:2. Abbreviations are Chymo, chymotrypsin; CnBr, cyanogen bromide; NH2OH, hydroxylamine; NTCB, NTCB (2-nitro-5-thiocyanobenzoic acid) +Ni; pH2.5, pH 2.5; ProEn, proline endopeptidase; Staph, Staphylococcal protease; Trypsin, trypsin.

Prior to setting forth the invention in detail, it may be helpful to the understanding thereof to define the following terms:

The term “affinity tag” is used herein to denote a polypeptide segment that can be attached to a second polypeptide to provide for purification of the second polypeptide or provide sites for attachment of the second polypeptide to a substrate. In principal, any peptide or protein for which an antibody or other specific binding agent is available can be used as an affinity tag. Affinity tags include a poly-histidine tract, protein A (Nilsson et al., EMBO J. 4:1075, 1985; Nilsson et al., Methods Enzymol. 198:3, 1991), glutathione S transferase (Smith and Johnson, Gene 67:31, 1988), Glu-Glu affinity tag (Grussenmeyer et al., Proc. Natl. Acad. Sci. USA 82:7952-4, 1985) (SEQ ID NO:7), substance P, Flag™ peptide (Hopp et al., Biotechnology 6:1204-1210, 1988), streptavidin binding peptide, maltose binding protein (Guan et al., Gene 67:21-30, 1987), cellulose binding protein, thioredoxin, ubiquitin, T7 polymerase, or other antigenic epitope or binding domain. See, in general, Ford et al., Protein Expression and Purification 2: 95-107, 1991. DNAs encoding affinity tags and other reagents are available from commercial suppliers (e.g., Pharmacia Biotech, Piscataway, N.J.; New England Biolabs, Beverly, Mass.; Eastman Kodak, New Haven, Conn.).

The term “allelic variant” is used herein to denote any of two or more alternative forms of a gene occupying the same chromosomal locus. Allelic variation arises naturally through mutation, and may result in phenotypic polymorphism within populations. Gene mutations can be silent (no change in the encoded polypeptide) or may encode polypeptides having altered amino acid sequences. The term allelic variant is also used herein to denote a protein encoded by an allelic variant of a gene.

The terms “amino-terminal” and “carboxyl-terminal” are used herein to denote positions within polypeptides. Where the context allows, these terms are used with reference to a particular sequence or portion of a polypeptide to denote proximity or relative position. For example, a certain sequence positioned carboxyl-terminal to a reference sequence within a polypeptide is located proximal to the carboxyl terminus of the reference sequence, but is not necessarily at the carboxyl terminus of the complete polypeptide.

A “beta-strand-like region” is a region of a protein characterized by certain combinations of the polypeptide backbone dihedral angles phi (φ) and psi (ψ). Regions wherein φ is less than −60° and ψ is greater than 90° are beta-strand-like. Those skilled in the art will recognize that the limits of a β-strand are somewhat imprecise and may vary with the criteria used to define them. See, for example, Richardson and Richardson in Fasman, ed., Prediction of Protein Structure and the Principles of Protein Conformation, Plenum Press, New York, 1989; and Lesk, Protein Architecture: A Practical Approach, Oxford University Press, New York, 1991.

A “complement” of a polynucleotide molecule is a polynucleotide molecule having a complementary base sequence and reverse orientation as compared to a reference sequence. For example, the sequence 5′ ATGCACGGG 3′ is complementary to 5′ CCCGTGCAT 3′.

“Conservative amino acid substitutions” are defined by the BLOSUM62 scoring matrix of Henikoff and Henikoff, Proc. Natl. Acad. Sci. USA 89:10915-10919, 1992, an amino acid substitution matrix derived from about 2,000 local multiple alignments of protein sequence segments, representing highly conserved regions of more than 500 groups of related proteins. As used herein, the term “conservative amino acid substitution” refers to a substitution represented by a BLOSUM62 value of greater than −1. For example, an amino acid substitution is conservative if the substitution is characterized by a BLOSUM62 value of 0, 1, 2, or 3. Preferred conservative amino acid substitutions are characterized by a BLOSUM62 value of at least one 1 (e.g., 1, 2 or 3), while more preferred conservative amino acid substitutions are characterized by a BLOSUM62 value of at least 2 (e.g., 2 or 3).

The term “corresponding to”, when applied to positions of amino acid residues in sequences, means corresponding positions in a plurality of sequences when the sequences are optimally aligned.

The term “degenerate nucleotide sequence” denotes a sequence of nucleotides that includes one or more degenerate codons (as compared to a reference polynucleotide molecule that encodes a polypeptide). Degenerate codons contain different triplets of nucleotides, but encode the same amino acid residue (i.e., GAU and GAC triplets each encode Asp).

The term “expression vector” is used to denote a DNA molecule, linear or circular, that comprises a segment encoding a polypeptide of interest operably linked to additional segments that provide for its transcription. Such additional segments include promoter and terminator sequences, and may also include one or more origins of replication, one or more selectable markers, an enhancer, a polyadenylation signal, etc. Expression vectors are generally derived from plasmid or viral DNA, or may contain elements of both.

An “inhibitory polynucleotide” is a DNA or RNA molecule that reduces or prevents expression (transcription or translation) of a second (target) polynucleotide. Inhibitory polynucleotides include antisense polynucleotides, ribozymes, and external guide sequences. The term “inhibitory polynucleotide” further includes DNA and RNA molecules that encode the actual inhibitory species, such as DNA molecules that encode ribozymes.

The term “isolated”, when applied to a polynucleotide, denotes that the polynucleotide has been removed from its natural genetic milieu and is thus free of other extraneous or unwanted coding sequences, and is in a form suitable for use within genetically engineered protein production systems. Such isolated molecules are those that are separated from their natural environment and include cDNA and genomic clones. Isolated DNA molecules of the present invention are free of other genes with which they are ordinarily associated, but may include naturally occurring 5′ and 3′ untranslated regions such as promoters and terminators. The identification of associated regions will be evident to one of ordinary skill in the art (see for example, Dynan and Tijan, Nature 316:774-78, 1985).

An “isolated” polypeptide or protein is a polypeptide or protein that is found in a condition other than its native environment, such as apart from blood and animal tissue. An isolated polypeptide or protein may be prepared substantially free of other polypeptides or proteins, particularly those of animal origin. When used in this context, the term “isolated” does not exclude the presence of the same polypeptide or protein in alternative physical forms, such as dimers or alternatively glycosylated or derivatized forms.

“Operably linked” means that two or more entities are joined together such that they function in concert for their intended purposes. When referring to DNA segments, the phrase indicates, for example, that coding sequences are joined in the correct reading frame, and transcription initiates in the promoter and proceeds through the coding segment(s) to the terminator. When referring to polypeptides, “operably linked” includes both covalently (e.g., by disulfide bonding) and non-covalently (e.g., by hydrogen bonding, hydrophobic interactions, or salt-bridge interactions) linked sequences, wherein the desired function(s) of the sequences are retained.

The term “ortholog” denotes a polypeptide or protein obtained from one species that is the functional counterpart of a polypeptide or protein from a different species. Sequence differences among orthologs are the result of speciation.

A “polynucleotide” is a single- or double-stranded polymer of deoxyribonucleotide or ribonucleotide bases read from the 5′ to the 3′ end. Polynucleotides include RNA and DNA, and may be isolated from natural sources, synthesized in vitro, or prepared from a combination of natural and synthetic molecules. Sizes of polynucleotides are expressed as base pairs (abbreviated “bp”), nucleotides (“nt”), or kilobases (“kb”). Where the context allows, the latter two terms may describe polynucleotides that are single-stranded or double-stranded. When the term is applied to double-stranded molecules it is used to denote overall length and will be understood to be equivalent to the term “base pairs”. It will be recognized by those skilled in the art that the two strands of a double-stranded polynucleotide may differ slightly in length and that the ends thereof may be staggered as a result of enzymatic cleavage; thus all nucleotides within a double-stranded polynucleotide molecule may not be paired. Such unpaired ends will in general not exceed 20 nt in length.

A “polypeptide” is a polymer of amino acid residues joined by peptide bonds, whether produced naturally or synthetically. Polypeptides of less than about 10 amino acid residues are commonly referred to as “peptides”.

The term “promoter” is used herein for its art-recognized meaning to denote a portion of a gene containing DNA sequences that provide for the binding of RNA polymerase and initiation of transcription. Promoter sequences are commonly, but not always, found in the 5′ non-coding regions of genes.

A “protein” is a macromolecule comprising one or more polypeptide chains. A protein may also comprise non-peptidic components, such as carbohydrate groups. Carbohydrates and other non-peptidic substituents may be added to a protein by the cell in which the protein is produced, and will vary with the type of cell. Proteins are defined herein in terms of their amino acid backbone structures; substituents such as carbohydrate groups are generally not specified, but may be present nonetheless. Thus, a protein “consisting of”, for example, from 15 to 1500 amino acid residues may further contain one or more carbohydrate chains.

The term “receptor” denotes a cell-associated protein that binds to a bioactive molecule (i.e., a ligand) and mediates the effect of the ligand on the cell.

Membrane-bound receptors are characterized by a multi-domain structure comprising an extracellular ligand-binding domain and an intracellular effector domain that is typically involved in signal transduction. Many cell-surface receptors are, in their active forms, multi-subunit structures in which the ligand-binding and signal transduction functions may reside in separate subunits. Binding of ligand to receptor results in a conformational change in the receptor that causes an interaction between the effector domain and other molecule(s) in the cell. This interaction in turn leads to an alteration in the metabolism of the cell. Metabolic events that are linked to receptor-ligand interactions include gene transcription, phosphorylation, dephosphorylation, increases in cyclic AMP production, mobilization of cellular calcium, mobilization of membrane lipids, cell adhesion, hydrolysis of inositol lipids, and hydrolysis of phospholipids. In general, receptors can be membrane bound, cytosolic or nuclear; monomeric (e.g., thyroid stimulating hormone receptor, beta-adrenergic receptor) or multimeric (e.g., PDGF receptor, growth hormone receptor, IL-3 receptor, GM-CSF receptor, G-CSF receptor, erythropoietin receptor and IL-6 receptor).

A “secretory signal sequence” is a DNA sequence that encodes a polypeptide (a “secretory peptide”) that, as a component of a larger polypeptide, directs the larger polypeptide through a secretory pathway of a cell in which it is synthesized.

The larger polypeptide is commonly cleaved to remove the secretory peptide during transit through the secretory pathway.

A “segment” is a portion of a larger molecule (e.g., polynucleotide or polypeptide) having specified attributes. For example, a DNA segment encoding a specified polypeptide is a portion of a longer DNA molecule, such as a plasmid or plasmid fragment, that, when read from the 5′ to the 3′ direction, encodes the sequence of amino acids of the specified polypeptide.

The term “splice variant” is used herein to denote alternative forms of RNA transcribed from a gene. Splice variation arises naturally through use of alternative splicing sites within a transcribed RNA molecule, or less commonly between separately transcribed RNA molecules, and may result in several mRNAs transcribed from the same gene. Splice variants may encode polypeptides having altered amino acid sequence. The term splice variant is also used herein to denote a protein encoded by a splice variant of an mRNA transcribed from a gene.

Molecular weights and lengths of polymers determined by imprecise analytical methods (e.g., gel electrophoresis) will be understood to be approximate values. When such a value is expressed as “about” X or “approximately” X, the stated value of X will be understood to be accurate to ±10%.

All references cited herein are incorporated by reference in their entirety.

The present invention is based in part upon the discovery of novel human and mouse polypeptides, designated “zcub5.” Modeling of the zcub5 amino acid sequence predicts the presence of four domains in the mature protein. Referring to SEQ ID NO:2, a CUB domain extends from residue 41 to residue 150. A factor V/VII domain extends from residue 258 to residue 412. A transmembrane domain extends from residue 453 to residue 479. An intracellular domain extends from residue 480 to residue 715. In addition, there is a predicted secretory peptide comprising residues 1-34 of SEQ ID NO:2. Those skilled in the art will recognize that these predicted domain boundaries are approximate and may vary by ±5 residues. Zcub5 shows significant homology to neuropilin-1. NP-1 contains 2 CUB domains, 2 factor V/VIII domains, a MAM domain, a transmembrane domain, and a cytoplasmic domain. As discussed above, NP-1 is an important VEGF receptor involved in cardiovascular development and binds to placenta growth factor (PlGF), various semaphorins (which inhibit growth of axons), and the VEGF receptor Flt-1 (Fuh et al., J. Biol. Chem. 275:26690-26695, 2000). On the basis of this homology it is believed that zcub5 will bind to members of the PDGF/VEGF and/or semaphorin families.

Within SEQ ID NO:4 (mouse zcub5 variant “x2”), the secretory peptide extends from residue 1 to residue 25, the CUB domain from residue 32 to residue 141, the transmembrane domain from residue 245 to residue 271, and the intracellular domain from residue 272 to residue 503. Within SEQ ID NO:6 (mouse zcub5 variant “x3”), the secretory peptide extends from residue 1 to residue 25, the CUB domain from residue 32 to residue 99, the transmembrane domain from residue 200 to residue 226, and the intracellular domain from residue 227 to residue 458. Because the CUB domain is truncated in the x3 variant, the protein is expected to have significantly diminished ligand-binding activity.

The mouse sequences shown in SEQ ID NOS:3-6 exhibit alternative splicing patterns in comparison to the human sequence (SEQ ID NOS:1 and 2). An alignment of the three polypeptide sequences is shown in FIGS. 2A-2B. Those skilled in the art will recognize that additional splicing variants of the human and mouse sequences are expected to occur. Naturally occuring, soluble forms of these proteins may also be found.

The CUB domain of zcub5 is homologous to CUB domains of Xenopus laevis neuropilin precursor (Takagi et al., ibid.), human BMP-1 (Wozney et al., ibid.), and X. laevis tolloid-like protein (Lin et al., ibid.). These CUB domains are 100-120 residues in length and are characterized by the presence of two conserved motifs, each of which contains a disulfide-bonded cysteine pair. The first motif conforms generally to the sequence C[Gsde][Gryst]X {6,10}[Gst]X[ILFVsy]X[STahn][Plai][NSedh][YFWG][Pig]X{3,5}[Yfsd]X{2,6}CX[WYkr]X[ILVf] (SEQ ID NO:8), wherein square brackets indicate the allowable residues at a given position, with upper case letters indicating more common residues; X indicates a variable residue; and X{y,z} is a block of variable residues from y to z residues in length. Within SEQ ID NO:2 this motif occurs at residues 41-72, with the conserved Cys residues at positions 41 and 68. The second motif conforms generally to the sequence C[KRGAilwp][YWKis][DE][WYFqsavi]X{11,15}[Gnem][KRivsp][WYFlim]CG (SEQ ID NO:9). Within SEQ ID NO:2 this motif occurs at residues 94-113, with the conserved Cys residues at positions 94 and 112. Cysteine pairs 41-68 and 94-112 are predicted to form disulfide bonds.

The predicted beta-barrel structure of the CUB domain comprises beta-strand-like regions comprising residues 43-46, 50-54, 67-73, 82-87, 89-91, 97-102, 107-111, 113-115, 117-120, 127-134, and 144-150 of human zcubS (SEQ ID NO:2).

Factor V/VIII domains also have two conserved regions, which are different parts of a single, functional domain. Factor V/VIII domains occur in a wide range of proteins, where they are believed to function in cell adhesion and/or phospholipid binding. Many of the proteins that contain this domain are also involved in some neuronal functions. See, PROSITE: http://www.expasy.ch/cgi-bin/nicedoc.pl?PDOC00988. The first conserved region can be represented by the sequence motif [GAS]Wx{7,15}[FYW][LIV]x[LIVFA][GSTDEN]xxxxxx[LIVF]xx[IV]x[LIVT][QKM]G (SEQ ID NO:10), corresponding to residues 298-331 of SEQ ID NO:2. The second conserved region can be represented by the sequence motif Px{8,10}[LM]Rx[GE][LIVP]xGC (SEQ ID NO:11), corresponding to residues 396-412 of SEQ ID NO:2.

The present invention provides polypeptides that comprise an epitope-bearing portion of a protein as shown in SEQ ID NO:2, SEQ ID NO:4, or SEQ ID NO:6. An “epitope” is a region of a protein to which an antibody can bind. See, for example, Geysen et al., Proc. Natl. Acad. Sci. USA 81:3998-4002, 1984. Epitopes can be linear or conformational, the latter being composed of discontinuous regions of the protein that form an epitope upon folding of the protein. Linear epitopes are generally at least 6 amino acid residues in length. Relatively short synthetic peptides that mimic part of a protein sequence are routinely capable of eliciting an antiserum that reacts with the partially mimicked protein. See, Sutcliffe et al., Science 219:660-666, 1983. The present invention thus provides polypeptides comprising at least 6 contiguous amino acid residues of SEQ ID NO:2, SEQ ID NO:4, or SEQ ID NO:6, optionally at least 9 or at least 15 contiguous amino acid residues, and, within certain embodiments of the invention, the polypeptides comprise 20, 30, 40, 50, 100, or more contiguous residues of SEQ ID NO:2, SEQ ID NO:4, or SEQ ID NO:6, up to the entire predicted mature polypeptide (e.g., residues 35 to 715 of SEQ ID NO:2) or the primary translation product (e.g., residues 1 to 715 of SEQ ID NO:2). Also included in the present invention are polypeptides comprising a CUB domain, factor V/VIII domain, transmembrane domain, or intracellular domain of a zcubS protein. As disclosed in more detail below, these polypeptides can further comprise additional, non-zcub5, polypeptide sequence(s).

Antigenic, epitope-bearing polypeptides of the present invention are useful for raising antibodies, including monoclonal antibodies, that specifically bind to a zcub5 protein. It is preferred that the amino acid sequence of the epitope-bearing polypeptide is selected to provide substantial solubility in aqueous solvents, that is the sequence includes relatively hydrophilic residues, and hydrophobic residues are substantially avoided. Such regions include those comprising residues 75-80, 222-227, 239-246, 305-311, 318-323, 351-357, 424-429, 433-439, 480-485, or 662-667 of SEQ ID NO:2; residues 66-71, 158-163, 232-238, 273-278, 313-319, or 354-361 of SEQ ID NO:4; and residues 66-71, 158-163, 187-193, 228-233, 268-274, or 309-316 of SEQ ID NO:6.

Antibodies that recognize short, linear epitopes are particularly useful in analytic and diagnostic applications that employ denatured protein, such as Western blotting (Tobin, Proc. Natl. Acad. Sci. USA 76:4350-4356, 1979), or in the analysis of fixed cells or tissue samples. Antibodies to linear epitopes are also useful for detecting fragments of zcub5, such as might occur in body fluids or cell culture media.

Polypeptides of the present invention can be prepared with one or more amino acid substitutions, deletions or additions as compared to SEQ ID NO:2. These changes are preferably of a minor nature, that is conservative amino acid substitutions and other changes that do not significantly affect the folding or activity of the protein or polypeptide, and include amino- or carboxyl-terminal extensions, such as an amino-terminal methionine residue, an amino or carboxyl-terminal cysteine residue to facilitate subsequent linking to maleimide-activated keyhole limpet hemocyanin, a small linker peptide of up to about 20-25 residues, or an extension that facilitates purification (an affinity tag) as disclosed above. Two or more affinity tags may be used in combination. Polypeptides comprising affinity tags can further comprise a polypeptide linker and/or a proteolytic cleavage site between the zcub5 polypeptide and the affinity tag. Exemplary cleavage sites include thrombin cleavage sites and factor Xa cleavage sites.

The proteins of the present invention can also comprise non-naturally occuring amino acid residues. Non-naturally occuring amino acids include, without limitation, trans-3-methylproline, 2,4-methanoproline, cis-4-hydroxyproline, trans-4-hydroxyproline, N-methylglycine, allo-threonine, methylthreonine, hydroxyethylcysteine, hydroxyethylhomocysteine, nitroglutamine, homoglutamine, pipecolic acid, tert-leucine, norvaline, 2-azaphenylalanine, 3-azaphenylalanine, 4-azaphenylalanine, and 4-fluorophenylalanine. Several methods are known in the art for incorporating non-naturally occuring amino acid residues into proteins. For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations is carried out in a cell-free system comprising an E. coli S30 extract and commercially available enzymes and other reagents. Proteins are purified by chromatography. See, for example, Robertson et al., J. Am. Chem. Soc. 113:2722, 1991; Ellman et al., Methods Enzymol. 202:301, 1991; Chung et al., Science 259:806-809, 1993; and Chung et al., Proc. Natl. Acad. Sci. USA 90:10145-10149, 1993). In a second method, translation is carried out in Xenopus oocytes by microinjection of mutated mRNA and chemically aminoacylated suppressor tRNAs (Turcatti et al., J. Biol. Chem. 271:19991-19998, 1996). Within a third method, E. Coli cells are cultured in the absence of a natural amino acid that is to be replaced (e.g., phenylalanine) and in the presence of the desired non-naturally occuring amino acid(s) (e.g., 2-azaphenylalanine, 3-azaphenylalanine, 4-azaphenylalanine, or 4-fluorophenylalanine). The non-naturally occuring amino acid is incorporated into the protein in place of its natural counterpart. See, Koide et al., Biochem. 33:7470-7476, 1994. Naturally occuring amino acid residues can be converted to non-naturally occuring species by in vitro chemical modification. Chemical modification can be combined with site-directed mutagenesis to further expand the range of substitutions (Wynn and Richards, Protein Sci. 2:395-403, 1993).

The present invention further provides a variety of other polypeptide fusions. For example, a zcub5 polypeptide can be prepared as a fusion to a dimerizing protein as disclosed in U.S. Pat. Nos. 5,155,027 and 5,567,584. Exemplary dimerizing proteins in this regard include immunoglobulin constant region domains. For example, the extracellular domains of a zcub5 polypeptide or a ligand-binding portion thereof (e.g., CUB domain, CUB domain +factor V/VIII domain, or factor V/VIII domain) can be prepared as a fusion with an IgG Fc fragment. The Fc fragment can be modified to alter effector functions and other properties associated with the native Ig. For example, amino acid substitutions can be made at EU index positions 234, 235, and 237 to reduce binding to FcγRI, and at EU index positions 330 and 331 to reduce complement fixation. See, Duncan et al., Nature 332:563-564, 1988; Winter et al., U.S. Pat. No. 5,624,821; Tao et al., J. Exp. Med. 178:661, 1993; and Canfield and Morrison, J. Exp. Med. 173:1483, 1991. The carboxyl-terminal lysine residue can be removed from the C_{H}3 domain to increase homogeneity of the product. The Cys residue within the hinge region that is ordinarily disulfide-bonded to the light chain can be replaced with another amino acid residue, such as a serine residue, if the Ig fusion is not co-expressed with a light chain polypeptide. Immunoglobulin-zcub5 polypeptide fusions can be expressed in genetically engineered cells to produce a variety of multimeric zcub5 analogs. In addition, a zcub5 polypeptide can be joined to another bioactive molecule, such as a cytokine, to provide a multi-functional molecule. One or more domains of a zcub5 polypeptide can be joined to a cytokine to enhance or otherwise modify its biological properties. Auxiliary domains can be fused to zcub5 polypeptides to target them to specific cells, tissues, or macromolecules (e.g., collagen).

For example, a zcub5 polypeptide or protein can be targeted to a predetermined cell type by fusing a zcub5 polypeptide to a ligand that specifically binds to a receptor on the surface of the target cell. In this way, polypeptides and proteins can be targeted for therapeutic or diagnostic purposes. A zcub5 polypeptide can be fused to two or more moieties, such as an affinity tag for purification and a targeting domain. Polypeptide fusions can also comprise one or more cleavage sites, particularly between domains. See, Tuan et al., Connective Tissue Research 34:1-9, 1996.

The present invention further provides polypeptide fusions comprising the zcub5 CUB domain. The CUB domain may be used to target other proteins to cells having cell-surface receptors that bind it. While not wishing to be bound by theory, the homology of zcub5 to neuropilin-1 suggests that the CUB domain may be used to target zcub5 or other proteins containing it to cells having cell-surface semaphorins, including mesenchymal cells (e.g., smooth muscle cells and fibroblasts), endothelial cells, neuronal cells, lymphocytes, and tumor cells. The zcub5 CUB domain can thus be joined to other moieties, including polypeptides (e.g., other growth factors, antibodies, and enzymes) and non-peptidic moieties (e.g., radionuclides, contrast agents, drugs, and the like), to target them to cells expressing cell-surface semaphorins. Cleavage sites can be provided between the CUB and other domains to allow for proteolytic release of the other domain through existing local proteases within tissues, or by proteases added from exogenous sources. The release of the targeted domain may provide more localized biological effects.

The CUB domain of zcub5 may also bind to extracellular matrix (ECM) components, allowing the use of zcub5 polypeptides comprising the CUB domain for targetting other moieties to the ECM. In this way peptidic and non-peptidic compounds can be localized to sites of ECM accumulation.

Polypeptide fusions of the present invention will generally contain not more than about 1,800 amino acid residues, often not more than about 1,500 residues, commonly not more than about 1,000 residues, and will in many cases be considerably smaller. For example, a zcub5 polypeptide of 450 residues (residues 1-450 of SEQ ID NO:2) can be fused to E. coli β-galactosidase (1,021 residues; see Casadaban et al., J. Bacteriol. 143:971-980, 1980), a 10-residue spacer, and a 4-residue factor Xa cleavage site to yield a polypeptide of 1,485 residues. In a second example, residues 1-715 of SEQ ID NO:2 can be fused to maltose binding protein (approximately 370 residues), a 4-residue cleavage site, and a 6-residue polyhistidine tag. In a third example, residues 1 to 150 of SEQ ID NO:2 are fused at the C terminus to an IgG Fc fragment of 232 residues to yield a primary translation product of 382 residues and a processed polypeptide of 348 residues.

Amino acid sequence changes are made in zcub5 polypeptides so as to minimize disruption of higher order structure essential to biological activity. Amino acid residues that are within regions or domains that are critical to maintaining structural integrity can be determined. Within these regions one can identify specific residues that will be more or less tolerant of change and maintain the overall tertiary structure of the molecule. Methods for analyzing sequence structure include, but are not limited to, alignment of multiple sequences with high amino acid or nucleotide identity, secondary structure propensities, binary patterns, complementary packing, and buried polar interactions (Barton, Current Opin. Struct. Biol. 5:372-376, 1995 and Cordes et al., Current Opin. Struct. Biol. 6:3-10, 1996). In general, determination of structure will be accompanied by evaluation of activity of modified molecules. For example, changes in amino acid residues will be made so as not to disrupt the beta barrel structure of the CUB domain. The effects of amino acid sequence changes can be predicted by, for example, computer modeling using available software (e.g., the Insight II® viewer and homology modeling tools; MSI, San Diego, Calif.) or determined by analysis of crystal structure (see, e.g., Lapthom et al, Nature 369:455-461, 1994; Lapthom et al., Nat. Struct. Biol. 2:266-268, 1995). Protein folding can be measured by circular dichroism (CD). Measuring and comparing the CD spectra generated by a modified molecule and standard molecule are routine in the art (Johnson, Proteins 7:205-214, 1990). Crystallography is another well known and accepted method for analyzing folding and structure. Nuclear magnetic resonance (NMR), digestive peptide mapping and epitope mapping are other known methods for analyzing folding and structural similarities between proteins and polypeptides (Schaanan et al., Science 257:961-964, 1992). Mass spectrometry and chemical modification using reduction and alkylation can be used to identify cysteine residues that are associated with disulfide bonds or are free of such associations (Bean et al., Anal. Biochem. 201:216-226, 1992; Gray, Protein Sci. 2:1732-1748, 1993; and Patterson et al., Anal. Chem. 66:3727-3732, 1994). Alterations in disulfide bonding will be expected to affect protein folding. These techniques can be employed individually or in combination to analyze and compare the structural features that affect folding of a variant protein or polypeptide to a standard molecule to determine whether such modifications would be significant.

A hydrophilicity profile of SEQ ID NO:2 is shown in FIG. 1. Those skilled in the art will recognize that this hydrophilicity will be taken into account when designing alterations in the amino acid sequence of a zcub5 polypeptide, so as not to disrupt the overall profile.

Essential amino acids in the polypeptides of the present invention can be identified experimentally according to procedures known in the art, such as site-directed mutagenesis or alanine-scanning mutagenesis (Cunningham and Wells, Science 244, 1081-1085, 1989; Bass et al., Proc. Natl. Acad. Sci. USA 88:4498-4502, 1991). In the latter technique, single alanine mutations are introduced throughout the molecule, and the resultant mutant molecules are tested for biological activity as disclosed below to identify amino acid residues that are critical to the activity of the molecule.

Multiple amino acid substitutions can be made and tested using known methods of mutagenesis and screening, such as those disclosed by Reidhaar-Olson and Sauer (Science 241:53-57, 1988) or Bowie and Sauer (Proc. Natl. Acad. Sci. USA 86:2152-2156, 1989). These authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30:10832-10837, 1991; Ladner et al., U.S. Pat. No. 5,223,409; Huse, WIPO Publication WO 92/06204) and region-directed mutagenesis (Derbyshire et al., Gene 46:145, 1986; Ner et al., DNA 7:127, 1988).

Variants of the disclosed zcub5 DNA and polypeptide sequences can be generated through DNA shuffling as disclosed by Stemmer, Nature 370:389-391, 1994 and Stemmer, Proc. Natl. Acad. Sci. USA 91:10747-10751, 1994. Briefly, variant genes are generated by in vitro homologous recombination by random fragmentation of a parent gene followed by reassembly using PCR, resulting in randomly introduced point mutations. This technique can be modified by using a family of parent genes, such as allelic variants or genes from different species, to introduce additional variability into the process. Selection or screening for the desired activity, followed by additional iterations of mutagenesis and assay provides for rapid “evolution” of sequences by selecting for desirable mutations while simultaneously selecting against detrimental changes.

In many cases, the structure of the final polypeptide product will result from processing of the nascent polypeptide chain by the host cell, thus the final sequence of a zcub5 polypeptide produced by a host cell will not always correspond to the full sequence encoded by the expressed polynucleotide. For example, expressing the complete zcub5 sequence in a cultured mammalian cell is expected to result in removal of at least the secretory peptide, while the same polypeptide produced in a prokaryotic host would not be expected to be cleaved. Differential processing of individual chains may result in heterogeneity of expressed polypeptides.

Mutagenesis methods as disclosed above can be combined with high volume or high-throughput screening methods to detect biological activity of zcub5 variant polypeptides. Assays that can be scaled up for high throughput include mitogenesis and receptor-binding assays, which can be run in a 96-well format. Mutagenesis of the CUB domain can be used to modulate its binding to target proteins, including members of the semaphorin and PDGF/VEGF families, including enhancing or inhibiting binding to selected family members. A modified spectrum of binding comprising a zcub5 CUB domain. Direct binding utilizing labeled CUB protein can be used to monitor changes in CUB domain binding activity to target proteins, which include proteins present in cell membranes and proteins present on cell surfaces. The CUB domain can be labeled by a variety of methods including radiolabeling with isotopes, such as ¹²⁵I, conjugation to enzymes such as alkaline phosphatase or horseradish peroxidase, conjugation with biotin, and conjugation with various fluorescent markers including FITC. These and other assays are disclosed in more detail below. Mutagenized DNA molecules that encode zcub5 polypeptides of interest can be recovered from the host cells and rapidly sequenced using modern equipment. These methods allow the rapid determination of the importance of individual amino acid residues in a polypeptide of interest, and can be applied to polypeptides of unknown structure.

Using the methods discussed above, one of ordinary skill in the art can prepare a variety of polypeptide fragments or variants of SEQ ID NO:2 that retain the activity of wild-type zcub5.

The present invention also provides zcub5 polynucleotide molecules. These polynucleotides include DNA and RNA, both single- and double-stranded, the former encompassing both the sense strand and the antisense strand. A representative DNA sequence encoding the amino acid sequence of SEQ ID NO:2 is shown in SEQ ID NO:1. Those skilled in the art will readily recognize that, in view of the degeneracy of the genetic code, considerable sequence variation is possible among these polynucleotide molecules. SEQ ID NO: 12 is a degenerate DNA sequence that encompasses all DNAs that encode the zcub5 polypeptide of SEQ ID NO: 2. Those skilled in the art will recognize that the degenerate sequence of SEQ ID NO:12 also provides all RNA sequences encoding SEQ ID NO:2 by substituting U for T. Thus, zcub5 polypeptide-encoding polynucleotides comprising nucleotides 1-2145 of SEQ ID NO:12 and their RNA equivalents are contemplated by the present invention, as are segments of SEQ ID NO:12 encoding other zcub5 polypeptides disclosed herein. Degenerate DNA sequences encoding the proteins of SEQ ID NO:4 and SEQ ID NO:6 are shown in SEQ ID NO:13 and SEQ ID NO:14, respectively. Table 1 sets forth the one-letter codes used within SEQ ID NOS:12, 13, and 14 to denote degenerate nucleotide positions. “Resolutions” are the nucleotides denoted by a code letter. “Complement” indicates the code for the complementary nucleotide(s). For example, the code Y denotes either C or T, and its complement R denotes A or G, A being complementary to T, and G being complementary to C.

TABLE 1

| Nucleotide | Resolutions | Complement | Resolutions |
|---|---|---|---|
| A | A | T | T |
| C | C | G | G |
| G | G | C | C |
| T | T | A | A |
| R | A\|G | Y | C\|T |
| Y | C\|T | R | A\|G |
| M | A\|C | K | G\|T |
| K | G\|T | M | A\|C |
| S | C\|G | S | C\|G |
| W | A\|T | W | A\|T |
| H | A\|C\|T | D | A\|G\|T |
| B | C\|G\|T | V | A\|C\|G |
| V | A\|C\|G | B | C\|G\|T |
| D | A\|G\|T | H | A\|C\|T |
| N | A\|C\|G\|T | N | A\|C\|G\|T |

The degenerate codons used in SEQ ID NOS:12, 13, and 14, encompassing all possible codons for a given amino acid, are set forth in Table 2, below.

TABLE 2

| | | | |
|---|---|---|---|
| Amino | One-Letter | | Degenerate |
| Acid | Code | Codons | Codon |

| | | | |
|---|---|---|---|
| Cys | C | TGC TGT | TGY |
| Ser | S | AGC AGT TCA TCC TCG TCT | WSN |
| Thr | T | ACA ACC ACG ACT | CAN |
| Pro | P | CCA CCC CCG CCT | CCN |
| Ala | A | GCA GCC GCG GCT | GCN |
| Gly | G | GGA GGC GGG GGT | GGN |
| Asn | N | AAC AAT | AAY |
| Asp | D | GAC GAT | GAY |
| Glu | E | GAA GAG | GAR |
| Gln | Q | CAA CAG | CAR |
| His | H | CAC CAT | CAY |
| Arg | R | AGA AGG CGA CGC CGG CGT | MGN |
| Lys | K | AAA AAG | AAR |
| Met | M | ATG | ATG |
| Ile | I | ATA ATC ATT | ATH |
| Leu | L | CTA CTC CTG CTT TTA TTG | YTN |
| Val | V | GTA GTC GTG GTT | GTN |
| Phe | F | TTC TTT | TTY |
| Tyr | Y | TAC TAT | TAY |
| Trp | W | TGG | TGG |
| Ter | . | TAA TAG TGA | TRR |
| Asn\|Asp | B | | RAY |
| Glu\|Gln | Z | | SAR |
| Any | X | | NNN |
| Gap | — | — | |

One of ordinary skill in the art will appreciate that some ambiguity is introduced in determining a degenerate codon, representative of all possible codons encoding each amino acid. For example, the degenerate codon for serine (WSN) can, in some circumstances, encode arginine (AGR), and the degenerate codon for arginine (MGN) can, in some circumstances, encode serine (AGY). A similar relationship exists between codons encoding phenylalanine and leucine. Thus, some polynucleotides encompassed by a degenerate sequence may encode variant amino acid sequences, but one of ordinary skill in the art can easily identify such variant sequences by reference to the amino acid sequence of SEQ ID NO: 2, SEQ ID NO:4, or SEQ ID NO:6. Variant sequences can be readily tested for functionality as described herein.

One of ordinary skill in the art will also appreciate that different species can exhibit preferential codon usage. See, in general, Grantham et al., Nuc. Acids Res. 8:1893-1912, 1980; Haas et al. Curr. Biol. 6:315-324, 1996; Wain-Hobson et al., Gene 13:355-364, 1981; Grosjean and Fiers, Gene 18:199-209, 1982; Holm, Nuc. Acids Res. 14:3075-3087, 1986; and Ikemura, J. Mol. Biol. 158:573-597, 1982. Introduction of preferred codon sequences into recombinant DNA can, for example, enhance production of the protein by making protein translation more efficient within a particular cell type or species. Therefore, the degenerate codon sequences disclosed in SEQ ID NOS:12, 13, and 14 serve as templates for optimizing expression of polynucleotides in various cell types and species commonly used in the art and disclosed herein.

Within certain embodiments of the invention the isolated polynucleotides will hybridize to similar sized regions of SEQ ID NO:1, 3, or 5, or a sequence complementary thereto, under stringent conditions. In general, stringent conditions are selected to be about 5° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Typical stringent conditions are those in which the salt concentration is up to about 0.03 M at pH 7 and the temperature is at least about 60° C.

As previously noted, the isolated polynucleotides of the present invention include DNA and RNA. Methods for preparing DNA and RNA are well known in the art. In general, RNA is isolated from a tissue or cell that produces large amounts of zcub5 RNA. Total RNA can be prepared using guanidine HCl extraction followed by isolation by centrifugation in a CsCl gradient (Chirgwin et al., Biochemistry 18:52-94, 1979). Poly (A)⁺ RNA is prepared from total RNA using the method of Aviv and Leder (Proc. Natl. Acad. Sci. USA 69:1408-1412, 1972). Complementary DNA (cDNA) is prepared from poly(A)⁺ RNA using known methods. In the alternative, genomic DNA can be isolated. Polynucleotides encoding zcub5 polypeptides are then identified and isolated by, for example, hybridization or PCR.

Full-length clones encoding zcub5 can be obtained by conventional cloning procedures. Complementary DNA (cDNA) clones are often preferred, although for some applications (e.g., expression in transgenic animals) it may be preferable to use a genomic clone, or to modify a cDNA clone to include at least one genomic intron. Methods for preparing cDNA and genomic clones are well known and within the level of ordinary skill in the art, and include the use of the sequence disclosed herein, or parts thereof, for probing or priming a library. Expression libraries can be probed with antibodies to zcub5, receptor fragments, or other specific binding partners.

Zcub5 polynucleotide sequences disclosed herein can also be used as probes or primers to clone 5′ non-coding regions of a zcub5 gene. Promoter elements from a zcub5 gene can be used to direct the expression of heterologous genes in, for example, transgenic animals or patients treated with gene therapy. Cloning of 5′ flanking sequences also facilitates production of zcub5 proteins by “gene activation” as disclosed in U.S. Pat. No. 5,641,670. Briefly, expression of an endogenous zcub5 gene in a cell is altered by introducing into the zcub5 locus a DNA construct comprising at least a targeting sequence, a regulatory sequence, an exon, and an unpaired splice donor site. The targeting sequence is a zcub5 5′ non-coding sequence that permits homologous recombination of the construct with the endogenous zcub5 locus, whereby the sequences within the construct become operably linked with the endogenous zcub5 coding sequence. In this way, an endogenous zcub5 promoter can be replaced or supplemented with other regulatory sequences to provide enhanced, tissue-specific, or otherwise regulated expression. SEQ ID NO: 15 shows approximately 1 kb of 5′-untranslated sequence from the human zcub5 gene, and is predicted to include the functional transcription promoter region. Nucleotides 999-1001 of SEQ ID NO: 15 are the initiation ATG codon.

Those skilled in the art will recognize that the sequences disclosed in SEQ ID NOS:1 and 2 represent a single allele of human zcub5 and the mouse sequences disclosed in SEQ ID NOS:3-6 represent splice variants, probably of a single allele. Allelic variants of these sequences can be cloned by probing cDNA or genomic libraries from different individuals according to standard procedures.

The present invention further provides counterpart polypeptides and polynucleotides from other species (“orthologs”). Of particular interest are zcub5 polypeptides from other mammalian species, including murine, porcine, ovine, bovine, canine, feline, equine, and other primate polypeptides. These non-human zcub5 polypeptides and polynucleotides, as well as antagonists thereof and other related molecules, can be used, inter alia, in veterinary medicine. Orthologs of human zcub5 can be cloned using information and compositions provided by the present invention in combination with conventional cloning techniques. For example, a cDNA can be cloned using mRNA obtained from a tissue or cell type that expresses zcub5 as disclosed above. A library is then prepared from mRNA of a positive tissue or cell line. A zcub5-encoding cDNA can then be isolated by a variety of methods, such as by probing with a complete or partial human cDNA or with one or more sets of degenerate probes based on the disclosed sequence. A cDNA can also be cloned using the polymerase chain reaction, or PCR (Mullis, U.S. Pat. No. 4,683,202), using primers designed from the representative human zcub5 sequence disclosed herein. Within an additional method, a cDNA library can be used to transform or transfect host cells, and expression of the DNA of interest can be detected with an antibody to zcub5 polypeptide. Similar techniques can also be applied to the isolation of genomic clones.

For any zcub5 polypeptide, including variants and fusion proteins, one of ordinary skill in the art can readily generate a fully degenerate polynucleotide sequence encoding that variant using the information set forth in Tables 1 and 2, above. Moreover, those of skill in the art can use standard software to devise zcub5 variants based upon the nucleotide and amino acid sequences described herein. The present invention thus provides a computer-readable medium encoded with a data structure that provides at least one of the following sequences: SEQ ID NO: 1, SEQ ID NO:2, SEQ ID NO:3, SEQ ID NO:4, SEQ ID NO:5, SEQ ID NO:6, SEQ ID NO:12, SEQ ID NO:13, SEQ ID NO:14, and portions thereof. Suitable forms of computer-readable media include, without limitation, a hard or fixed drive, a random access memory (RAM) chip, a floppy disk, digital linear tape (DLT), a disk cache, a ZIP™ disk, compact discs (e.g., CD-read only memory (ROM), CD-rewritable (RW), and CD-recordable), digital versatile/video discs (DVD) (e.g., DVD−ROM, DVD−RAM, and DVD+RW), and carrier waves.

The zcub5 polypeptides of the present invention, including full-length polypeptides, biologically active fragments, and fusion polypeptides can be produced according to conventional techniques using cells into which have been introduced an expression vector encoding the polypeptide. As used herein, “cells into which have been introduced an expression vector” include both cells that have been directly manipulated by the introduction of exogenous DNA molecules and progeny thereof that contain the introduced DNA. Suitable host cells are those cell types that can be transformed or transfected with exogenous DNA and grown in culture, and include bacteria, fungal cells, and cultured higher eukaryotic cells. Techniques for manipulating cloned DNA molecules and introducing exogenous DNA into a variety of host cells are disclosed by Sambrook et al., Molecular Cloning: A Laboratory Manual, 2nd ed., Cold Spring Harbor Laboratory Press, Cold Spring Harbor, N.Y., 1989, and Ausubel et al., eds., Current Protocols in Molecular Biology, John Wiley and Sons, Inc., NY, 1987.

In general, a DNA sequence encoding a zcub5 polypeptide is operably linked to other genetic elements required for its expression, generally including a transcription promoter and terminator, within an expression vector. The vector will also commonly contain one or more selectable markers and one or more origins of replication, although those skilled in the art will recognize that within certain systems selectable markers can be provided on separate vectors, and replication of the exogenous DNA is provided by integration into the host cell genome. Selection of promoters, terminators, selectable markers, vectors and other elements is a matter of routine design within the level of ordinary skill in the art. Many such elements are described in the literature and are available through commercial suppliers. See, in general, WO 00/34474.

To direct a zcub5 polypeptide into the secretory pathway of a host cell, a secretory signal sequence (also known as a leader sequence, prepro sequence or pre sequence) is provided in the expression vector. The secretory signal sequence may be that of zcub5, or may be derived from another secreted protein (e.g., t-PA; see, U.S. Pat. No. 5,641,655) or synthesized de novo. The secretory signal sequence is operably linked to the zcub5 DNA sequence, i.e., the two sequences are joined in the correct reading frame and positioned to direct the newly sythesized polypeptide into the secretory pathway of the host cell. Secretory signal sequences are commonly positioned 5′ to the DNA sequence encoding the polypeptide of interest, although certain signal sequences may be positioned elsewhere in the DNA sequence of interest (see, e.g., Welch et al., U.S. Pat. No. 5,037,743; Holland et al., U.S. Pat. No. 5,143,830).

Cultured mammalian cells can be used as hosts within the present invention. Methods for introducing exogenous DNA into mammalian host cells include calcium phosphate-mediated transfection (Wigler et al., Cell 14:725, 1978; Corsaro and Pearson, Somatic Cell Genetics 7:603, 1981; Graham and Van der Eb, Virology 52:456, 1973), electroporation (Neumann et al., EMBO J. 1:841-845, 1982), DEAE-dextran mediated transfection (Ausubel et al., ibid.), and liposome-mediated transfection (Hawley-Nelson et al., Focus 15:73, 1993; Ciccarone et al., Focus 15:80, 1993). The production of recombinant polypeptides in cultured mammalian cells is disclosed by, for example, Levinson et al., U.S. Pat. No. 4,713,339; Hagen et al., U.S. Pat. No. 4,784,950; Palmiter et al., U.S. Pat. No. 4,579,821; and Ringold, U.S. Pat. No. 4,656,134. Suitable cultured mammalian cells include the COS-1 (ATCC No. CRL 1650), COS-7 (ATCC No. CRL 1651), BHK (ATCC No. CRL 1632), BHK 570 (ATCC No. CRL 10314), 293 (ATCC No. CRL 1573; Graham et al., J. Gen. Virol. 36:59-72, 1977) and Chinese hamster ovary (e.g. CHO-K1, ATCC No. CCL 61; or CHO DG44, Chasin et al., Som. Cell. Molec. Genet. 12:555, 1986) cell lines. Additional suitable cell lines are known in the art and available from public depositories such as the American Type Culture Collection, Manassas, Va. Strong transcription promoters include promoters from SV-40 or cytomegalovirus. See, e.g., U.S. Pat. No. 4,956,288. Other suitable promoters include those from metallothionein genes (U.S. Pat. Nos. 4,579,821 and 4,601,978) and the adenovirus major late promoter. Expression vectors for use in mammalian cells include pZP-1 and pZP-9, which have been deposited with the American Type Culture Collection, Manassas, Va. USA under accession numbers 98669 and 98668, respectively, and derivatives thereof.

The adenovirus system (disclosed in more detail below) can also be used for protein production in vitro. By culturing adenovirus-infected non-293 cells under conditions where the cells are not rapidly dividing, the cells can produce proteins for extended periods of time. For instance, BHK cells are grown to confluence in cell factories, then exposed to the adenoviral vector encoding the secreted protein of interest. The cells are then grown under serum-free conditions, which allows infected cells to survive for several weeks without significant cell division. In an alternative method, adenovirus vector-infected 293 cells can be grown as adherent cells or in suspension culture at relatively high cell density to produce significant amounts of protein (See Garnier et al., Cytotechnol. 15:145-55, 1994). With either protocol an expressed, secreted heterologous protein can be repeatedly isolated from the cell culture supernatant, lysate, or membrane fractions depending on the disposition of the expressed protein in the cell. Within the infected 293 cell production protocol, non-secreted proteins can also be effectively obtained.

Insect cells can be infected with recombinant baculovirus, commonly derived from Autographa californica nuclear polyhedrosis virus (AcNPV) according to methods known in the art, such as the transposon-based system described by Luckow et al. (J. Virol. 67:4566-4579, 1993). This system, which utilizes transfer vectors, is commercially available in kit form (Bac-to-Bac™ kit; Life Technologies, Rockville, Md.). The transfer vector (e.g., pFastBac1™; Life Technologies) contains a Tn7 transposon to move the DNA encoding the protein of interest into a baculovirus genome maintained in E. coli as a large plasmid called a “bacmid.” See, Hill-Perkins and Possee, J. Gen. Virol. 71:971-976, 1990; Bonning et al., J. Gen. Virol. 75:1551-1556, 1994; and Chazenbalk and Rapoport, J. Biol. Chem. 270:1543-1549, 1995. In addition, transfer vectors can include an in-frame fusion with DNA encoding a polypeptide extension or affinity tag as disclosed above. Using techniques known in the art, a transfer vector containing a zcub5-encoding sequence is transformed into E. coli host cells, and the cells are screened for bacmids which contain an interrupted lacZ gene indicative of recombinant baculovirus. The bacmid DNA containing the recombinant baculovirus genome is isolated, using common techniques, and used to transfect Spodoptera frugiperda cells, such as Sf9 cells. Recombinant virus that expresses zcub5 protein is subsequently produced. Recombinant viral stocks are made by methods commonly used the art.

For protein production, the recombinant virus is used to infect host cells, typically a cell line derived from the fall armyworm, Spodoptera frugiperda (e.g., Sf9 or Sf21 cells) or Trichoplusia ni (e.g., High Five™ cells; Invitrogen, Carlsbad, Calif.).

See, for example, U.S. Pat. No. 5,300,435. Serum-free media are used to grow and maintain the cells. Suitable media formulations are known in the art and can be obtained from commercial suppliers. The cells are grown up from an inoculation density of approximately 2−5×10⁵ cells to a density of 1−2×10⁶ cells, at which time a recombinant viral stock is added at a multiplicity of infection (MOI) of 0.1 to 10, more typically near 3. Procedures used are generally known in the art.

Other higher eukaryotic cells can also be used as hosts, including plant cells and avian cells. The use of Agrobacterium rhizogenes as a vector for expressing genes in plant cells has been reviewed by Sinkar et al., J. Biosci. (Bangalore) 11:47-58, 1987.

Fungal cells, including yeast cells, can also be used within the present invention. Yeast species of particular interest in this regard include Saccharomyces cerevisiae, Pichia pastoris, and Pichia methanolica. Methods for transforming S. cerevisiae cells with exogenous DNA and producing recombinant polypeptides therefrom are disclosed by, for example, Kawasaki, U.S. Pat. No. 4,599,311; Kawasaki et al., U.S. Pat. No. 4,931,373; Brake, U.S. Pat. No. 4,870,008; Welch et al., U.S. Pat. No. 5,037,743; and Murray et al., U.S. Pat. No. 4,845,075. Transformed cells are selected by phenotype determined by the selectable marker, commonly drug resistance or the ability to grow in the absence of a particular nutrient (e.g., leucine). An exemplary vector system for use in Saccharomyces cerevisiae is the POT1 vector system disclosed by Kawasaki et al. (U.S. Pat. No. 4,931,373), which allows transformed cells to be selected by growth in glucose-containing media. Suitable promoters and terminators for use in yeast include those from glycolytic enzyme genes (see, e.g., Kawasaki, U.S. Pat. No. 4,599,311; Kingsman et al., U.S. Pat. No. 4,615,974; and Bitter, U.S. Pat. No. 4,977,092) and alcohol dehydrogenase genes. See also U.S. Pat. Nos. 4,990,446; 5,063,154; 5,139,936 and 4,661,454. Transformation systems for other yeasts, including Hansenula polymorpha, Schizosaccharomyces pombe, Kluyveromyces lactis, Kluyveromyces fragilis, Ustilago maydis, Pichia pastoris, Pichia methanolica, Pichia guillermondii and Candida maltosa are known in the art. See, for example, Gleeson et al., J. Gen. Microbiol. 132:3459-3465, 1986; Cregg, U.S. Pat. No. 4,882,279; and Raymond et al., Yeast 14, 11-23, 1998. Aspergillus cells can be utilized according to the methods of McKnight et al., U.S. Pat. No. 4,935,349. Methods for transforming Acremonium chrysogenum are disclosed by Sumino et al., U.S. Pat. No. 5,162,228. Methods for transforming Neurospora are disclosed by Lambowitz, U.S. Pat. No. 4,486,533. Production of recombinant proteins in Pichia methanolica is disclosed in U.S. Pat. Nos. 5,716,808, 5,736,383, 5,854,039, and 5,888,768.

Prokaryotic host cells, including strains of the bacteria Escherichia coli,Bacillus and other genera are also useful host cells within the present invention. Techniques for transforming these hosts and expressing foreign DNA sequences cloned therein are well known in the art (see, e.g., Sambrook et al., ibid.). When expressing a zcub5 polypeptide in bacteria such as E. coli, the polypeptide may be retained in the cytoplasm or may be directed to the periplasmic space by a bacterial secretion sequence. In the former case, the cells are lysed, and the zcub5 polypeptide is recovered from the lysate. If the polypeptide is present in the cytoplasm in insoluble granules, the cells are lysed and the granules are recovered and denatured using, for example, guanidine isothiocyanate or urea. The denatured polypeptide can then be refolded and dimerized by diluting the denaturant, such as by dialysis against a solution of urea and a combination of reduced and oxidized glutathione, followed by dialysis against a buffered saline solution. In the latter case, the polypeptide can be recovered from the periplasmic space in a soluble and functional form by disrupting the cells (by, for example, sonication or osmotic shock) to release the contents of the periplasmic space and recovering the protein, thereby obviating the need for denaturation and refolding.

Transformed or transfected host cells are cultured according to conventional procedures in a culture medium containing nutrients and other components required for the growth of the chosen host cells. A variety of suitable media, including defined media and complex media, are known in the art and generally include a carbon source, a nitrogen source, essential amino acids, vitamins and minerals. Media may also contain such components as growth factors or serum, as required. The growth medium will generally select for cells containing the exogenously added DNA by, for example, drug selection or deficiency in an essential nutrient which is complemented by the selectable marker carried on the expression vector or co-transfected into the host cell. Liquid cultures are provided with sufficient aeration by conventional means, such as shaking of small flasks or sparging of fermentors.

Within certain embodiments of the invention the zcub5 polypeptides and proteins are purified to ≧80% purity, to ≧90% purity, or to ≧95% purity. In some embodiments the polypeptides and proteins are prepared in a pharmaceutically pure state, that is, greater than 99.9% pure with respect to contaminating macromolecules, particularly other proteins and nucleic acids, and free of infectious and pyrogenic agents.

Expressed recombinant zcub5 proteins (including chimeric polypeptides and multimeric proteins) are purified by conventional protein purification methods, typically by a combination of chromatographic techniques. See, in general, Affinity Chromatography: Principles & Methods, Pharmacia LKB Biotechnology, Uppsala, Sweden, 1988; and Scopes, Protein Purification: Principles and Practice, Springer-Verlag, New York, 1994. Proteins comprising a polyhistidine affinity tag (typically about 6 histidine residues) can be purified by affinity chromatography on a nickel chelate resin. See, for example, Houchuli et al., Bio/Technol. 6: 1321-1325, 1988. Proteins comprising a glu-glu tag can be purified by immunoaffinity chromatography according to conventional procedures. See, for example, Grussenmeyer et al., ibid. Maltose binding protein fusions are purified on an amylose column according to methods known in the art.

Zcub5 polypeptides can also be prepared through chemical synthesis according to methods known in the art, including exclusive solid phase synthesis, partial solid phase methods, fragment condensation or classical solution synthesis. See, for example, Merrifield, J. Am. Chem. Soc. 85:2149, 1963; Stewart et al., Solid Phase Peptide Synthesis (2nd edition), Pierce Chemical Co., Rockford, Ill., 1984; Bayer and Rapp, Chem. Pept. Prot. 3:3, 1986; and Atherton et al., Solid Phase Peptide Synthesis: A Practical Approach, IRL Press, Oxford, 1989. In vitro synthesis is particularly advantageous for the preparation of smaller polypeptides.

Using methods known in the art, zcub5 proteins can be prepared as monomers or multimers; glycosylated or non-glycosylated; pegylated or non-pegylated; and may or may not include an initial methionine amino acid residue. Zcub5 proteins can be produced in soluble or membrane-bound forms. Soluble forms are those lacking the transmembrane domain and include, for example, polypeptides consisting of residues 41-452, 35-452, 1-452, 258-412, 41-412, 35-412, 1-412, 41-150, 35-150, or 1-150 of SEQ ID NO:2, as well as intermediate forms and fusion proteins (e.g., Ig Fc fusions) comprising these polypeptides.

Target cells for use in zcub5 activity assays include, without limitation, endothelial cells, smooth muscle cells, fibroblasts, pericytes, mesangial cells, liver stellate cells, neuronal cells (including glial cells, dendritic cells, and neurons), immune cells (including T-cells, B-cells, and monocytes), and tumor cells.

Zcub5 proteins are characterized by their activity, that is, their ability to bind to members of the PDGF/VEGF family of growth factors and/or to semaphorins. As such, soluble zcub5 proteins are expected to act as antagonists of their ligands by binding to those ligands and thereby preventing normal ligand-receptor interations. Cell-surface zcub5 proteins are expected to function as receptors and mediate the action of their ligands on cells. Inhibition of growth factor or semaphorin activity in vivo may be manifested as modulation of immune functions, angiogenesis, neurite growth or development, bone growth, tumor growth or metastasis, ischemic events, or other physiological processes.

Biological activity of zcub5 proteins is assayed using in vitro or in vivo assays designed to detect growth factor or semaphorin activity. Many suitable assays are known in the art, and representative assays are disclosed herein. Assays using cultured cells are most convenient for screening, such as for determining the effects of amino acid substitutions, deletions, or insertions. However, in view of the complexity of developmental processes (e.g., angiogenesis and vasculogenesis), in vivo assays will generally be employed to confirm and further characterize biological activity. Certain in vitro models, such as gel matrix models, are sufficiently complex to assay histological effects. Assays can be performed using exogenously produced proteins (including zcub5 fragments and fusion proteins), or may be carried out in vivo or in vitro using cells expressing the polypeptide(s) of interest. Representative assays are disclosed below.

The effects of zcub5 proteins on growth factor-induced angiogenesis can be measured using assays that are known in the art. In general, a zcub5 protein is assayed by adding the protein to a test system that responds to a growth factor. Anti-angiogenic activity of zcub5 is indicated by a reduction in growth factor-induced angiogenesis or an associated biological response. For example, the effect of zcub5 proteins on primordial endothelial cells in angiogenesis can be assayed in the chick chorioallantoic membrane angiogenesis assay (Leung, Science 246:1306-1309, 1989; Ferrara, Ann. NY Acad. Sci. 752:246-256, 1995). Briefly, a small window is cut into the shell of an eight-day old fertilized egg, and a test substance is applied to the chorioallantoic membrane. After 72 hours, the membrane is examined for neovascularization. Other suitable assays include microinjection of early stage quail (Coturnix coturnix japonica) embryos as disclosed by Drake et al. (Proc. Natl. Acad. Sci. USA 92:7657-7661, 1995). Induction of vascular permeability, which is indicative of angiogenic activity, is measured in assays designed to detect leakage of protein from the vasculature of a test animal (e.g., mouse or guinea pig) after administration of a test compound (Miles and Miles, J. Physiol. 118:228-257, 1952; Feng et al., J. Exp. Med. 183:1981-1986, 1996). In vitro assays for angiogenic activity include the tridimensional collagen gel matrix model (Pepper et al. Biochem. Biophys. Res. Comm. 189:824-831, 1992 and Ferrara et al., Ann. NY Acad. Sci. 732:246-256, 1995), which measures the formation of tube-like structures by microvascular endothelial cells; and matrigel models (Grant et al., “Angiogenesis as a component of epithelial-mesenchymal interactions” in Goldberg and Rosen, Epithelial-Mesenchymal Interaction in Cancer, Birkhäuser Verlag, 1995, 235-248; Baatout, Anticancer Research 17:451-456, 1997), which are used to determine effects on cell migration and tube formation by endothelial cells seeded in matrigel, a basement membrane extract enriched in laminin.

Binding of zcub5 proteins to growth factors or other ligands can be measured using assays that detect a bound complex. Many such assays are known in the art and include immunological assays such as ELISA and sandwich assays. Immobilized zcub5 protein or immobilized growth factor can be used to capture the other partner. Receptor binding assays can be used to measure the ability of a zcub5 protein to modulate binding of a growth factor or other protein to its receptor. Such assays can be performed on cell lines that contain known cell-surface receptors for evaluation. The receptors can be naturally present in the cell, or can be recombinant receptors expressed by genetically engineered cells. See, for example, Bowen-Pope and Ross, Methods Enzymol. 109:69-100, 1985. Receptor binding can also be determined using a commercially available biosensor instrument (BIAcore™, Pharmacia Biosensor, Piscataway, N.J.), wherein protein is immobilized onto the surface of a receptor chip. See, Karlsson, J. Immunol. Methods 145:229-240, 1991 and Cunningham and Wells, J. Mol. Biol. 234:554-563, 1993. This system allows the determination of on- and off-rates, from which binding affinity can be calculated, and assessment of stoichiometry of binding.

Binding of zcub5 proteins to semaphorins can be assayed using isolated semaphorins or cells expressing cell-surface semaphorins. For example, cultured mammalian cells (e.g., COS cells) can be transfected to express cell-surface semaphorins and used to detect binding of labeled zcub5 proteins. Binding to soluble semaphorins can be assayed using conventional methods, including immunological assays, as disclosed above.

Zcub5-induced modulation of mitogenic activity can be measured using known assays, including ³H-thymidine incorporation assays (as disclosed by, e.g., Raines and Ross, Methods Enzymol. 109:749-773, 1985 and Wahl et al., Mol. Cell Biol. 8:5016-5025, 1988), dye incorporation assays (as disclosed by, for example, Mosman, J. Immunol. Meth. 65:55-63, 1983 and Raz et al., Acta Trop. 68:139-147, 1997) or cell counts. Suitable mitogenesis assays measure incorporation of ³H-thymidine into 20% confluent cultures or quiescent cells held at confluence for 48 hours. Suitable dye incorporation assays include measurement of the incorporation of the dye Alamar blue (Raz et al., ibid.) into target cells. See also, Gospodarowicz et al., J. Cell. Biol. 70:395-405, 1976; Ewton and Florini, Endocrinol. 106:577-583, 1980; and Gospodarowicz et al., Proc. Natl. Acad. Sci. USA 86:7311-7315, 1989.

Zcub5 activity can also be measured using assays that measure axon guidance and growth, which can be used to detect the modulation of axon outgrowth by zcub5 in the presence and absence of other bioactive agents (e.g., semaphorins or growth factors). Of particular interest are assays that indicate changes in neuron growth patterns, for example those disclosed in Hastings, WIPO Publication WO 97/29189 and Walter et al., Development 101:685-696, 1987. Assays to measure the effects on neuron growth are well known in the art. For example, the C assay (e.g., Raper and Kapfhammer, Neuron 4:21-29, 1990 and Luo et al., Cell 75:217-227, 1993) can be used to determine inhibition by zcub5 of the collapsing activity of semaphorins on growing neurons. Other methods that can assess zcub5-induced effects on neurite extension are also known. See, Goodman, Annu. Rev. Neurosci. 19:341-377, 1996. Conditioned media from cells expressing a zcub5 protein, a zcub5 agonist, or a zcub5 antagonist, or aggregates of such cells, can by placed in a gel matrix near suitable neural cells, such as dorsal root ganglia (DRG) or sympathetic ganglia explants, which have been co-cultured with nerve growth factor. Compared to control cells, zcub5-induced changes in neuron growth can be measured as disclosed by, for example, Messersmith et al., Neuron 14:949-959, 1995 and Puschel et al., Neuron 14:941-948, 1995. See also, Kitsukawa et al., Neuron 19:995-1005, 1997. Neurite outgrowth can also be measured using neuronal cell suspensions. See, for example, O'Shea et al., Neuron 7:231-237, 1991 and DeFreitas et al., Neuron 15:333-343, 1995. These assays can be used, for example, to measure the inhibition by zcub5 of semaphorin-induced growth cone collapse.

Monocyte activation assays are carried out to determine the ability of zcub5 proteins to modulate monocyte activation (Fuhlbrigge et al., J. Immunol. 138:

3799-3802, 1987). IL-1 and TNF(x levels produced in response to activation are measured by ELISA (reagents available from Biosource, Inc. Camarillo, Calif.). Monocyte/macrophage cells, by virtue of CD14 (LPS receptor), are exquisitely sensitive to endotoxin, and proteins with moderate levels of endotoxin-like activity will activate these cells. Monocytes can be cultured in the presence of one or more test substances (for example, a semaphorin +/− a zcub5 protein) for twenty hours, at which time monocyte aggregation is indicative of activation.

Soluble forms of zcub5 comprising the factor V/VIII domain can be assayed for the ability to modulate blood coagulation. Blood coagulation and chromogenic assays, which can be used to detect procoagulant, anticoagulant, and thrombolytic activities, are known in the art. For example, pro- and anticoagulant activities can be measured in a one-stage clotting assay using platelet-poor or factor-deficient plasma (Levy and Edgington, J. Exp. Med. 151:1232-1243, 1980; Schwartz et al., J. Clin. Invest. 67:1650-1658, 1981). The inhibition of coagulation factors can be measured using chromogenic substrates or in conventional coagulation assays (e.g., clotting time of normal human plasma; Dennis et al., J. Biol. Chem. 270:25411-25417, 1995). Activation of thrombin can be determined by hydrolysis of peptide p-nitroanilide substrates as disclosed by Lottenberg et al. (Thrombosis Res. 28:313-332, 1982). Factor VIII activity is assayed in a chromogenic assay that measures the factor VIII-dependent generation of factor Xa from factor X (Kabi Coatest method). Other procoagulant, anticoagulant, and thrombolytic activities can be measured using appropriate chromogenic substrates, a variety of which are available from commercial suppliers. See, for example, Kettner and Shaw, Methods Enzymol. 80:826-842, 1981.

The activity of zcub5 proteins can be measured with a silicon-based biosensor microphysiometer that measures the extracellular acidification rate or proton excretion associated with physiologic cellular responses to growth factors or semaphorins. An exemplary such device is the Cytosensor™ Microphysiometer manufactured by Molecular Devices, Sunnyvale, Calif. A variety of cellular responses, such as cell proliferation, ion transport, energy production, inflammatory response, regulatory and receptor activation, and the like, can be measured by this method. See, for example, McConnell et al., Science 257:1906-1912, 1992; Pitchford et al., Meth. Enzymol. 228:84-108, 1997; Arimilli et al., J. Immunol. Meth. 212:49-59, 1998; and Van Liefde et al., Eur. J. Pharmacol. 346:87-95, 1998. The microphysiometer can be used for assaying adherent or non-adherent eukaryotic or prokaryotic cells. By measuring extracellular acidification changes in cell media over time, the microphysiometer directly measures cellular responses to various stimuli. A microphysiometer can thus be used to detect zcub5-mediated inhibition of growth factor or semaphorin activity on responsive cells. In general, a first portion of cells responsive to a growth factor or semaphorin are cultured in the presence of the growth factor or semaphorin, and a second portion of the cells are cultured in the presence of the growth factor or semaphorin in combination with a zcub5 protein. A reduction in a cellular response of the second portion of the cells as compared to the first portion of the cells indicates inhibition of growth factor or semphorin activity by the zcub5 protein.

The biological activities of zcub5 proteins can be studied in non-human animals by administration of exogenous protein, by expression of zcub5-encoding polynucleotides, and by suppression of endogenous zcub5 expression through the use of inhibitory polynucleotides or knock-out techniques. Test animals are monitored for changes in such parameters as clinical signs, body weight, blood cell counts, clinical chemistry, histopathology, and the like. For example, stimulation of coronary collateral growth can be measured in known animal models, including a rabbit model of peripheral limb ischemia and hind limb ischemia and a pig model of chronic myocardial ischemia (Ferrara et al., Endocrine Reviews 18:4-25, 1997). These models can be modified by the use of adenovirus or naked DNA for gene delivery as disclosed in more detail below, resulting in local expression of the test protein(s). Angiogenic activity can also be tested in a rodent model of corneal neovascularization as disclosed by Muthukkaruppan and Auerbach, Science 205:1416-1418, 1979, wherein a test substance is inserted into a pocket in the cornea of an inbred mouse. For use in this assay, proteins are combined with a solid or semi-solid, biocompatible carrier, such as a polymer pellet. Angiogenesis is followed microscopically. Vascular growth into the corneal stroma can be detected in about 10 days. Angiogenic activity can also be tested in the hampster cheek pouch assay (Hockel et al., Arch. Surg. 128:423-429, 1993). A test substance is injected subcutaneously into the cheek pouch, and after five days the pouch is examined under low magnification to determine the extent of neovascularization. Tissue sections can also be examined histologically. Induction of vascular permeability is measured in assays designed to detect leakage of protein from the vasculature of a test animal (e.g., mouse or guinea pig) after administration of a test compound (Miles and Miles, J. Physiol. 118:228-257, 1952; Feng et al., J. Exp. Med. 183:1981-1986, 1996).

One in vivo approach for assaying proteins of the present invention utilizes viral delivery systems. Exemplary viruses for this purpose include adenovirus, herpesvirus, retroviruses, vaccinia virus, and adeno-associated virus (AAV). Adenovirus, a double-stranded DNA virus, is currently the best studied gene transfer vector for delivery of heterologous nucleic acids. For review, see Becker et al., Meth. Cell Biol. 43:161-89, 1994; and Douglas and Curiel, Science & Medicine 4:44-53, 1997. The adenovirus system offers several advantages. Adenovirus can (i) accommodate relatively large DNA inserts; (ii) be grown to high-titer; (iii) infect a broad range of mammalian cell types; and (iv) be used with many different promoters including ubiquitous, tissue specific, and regulatable promoters. Because adenoviruses are stable in the bloodstream, they can be administered by intravenous injection.

By deleting portions of the adenovirus genome, larger inserts (up to 7 kb) of heterologous DNA can be accommodated. These inserts can be incorporated into the viral DNA by direct ligation or by homologous recombination with a co-transfected plasmid. In an exemplary system, the essential E1 gene is deleted from the viral vector, and the virus will not replicate unless the E1 gene is provided by the host cell (e.g., the human 293 cell line). When intravenously administered to intact animals, adenovirus primarily targets the liver. If the adenoviral delivery system has an El gene deletion, the virus cannot replicate in the host cells. However, the host's tissue (e.g., liver) will express and process (and, if a signal sequence is present, secrete) the heterologous protein. Secreted proteins will enter the circulation in the highly vascularized liver, and effects on the infected animal can be determined.

An alternative method of gene delivery comprises removing cells from the body and introducing a vector into the cells as a naked DNA plasmid. The transformed cells are then re-implanted in the body. Naked DNA vectors are introduced into host cells by methods known in the art, including transfection, electroporation, microinjection, transduction, cell fusion, DEAE dextran, calcium phosphate precipitation, use of a gene gun, or use of a DNA vector transporter. See, Wu et al., J. Biol. Chem. 263:14621-14624, 1988; Wu et al., J. Biol. Chem. 267:963-967, 1992; and Johnston and Tang, Meth. Cell Biol. 43:353-365, 1994.

Transgenic mice, engineered to express a zcub5 gene, and mice that exhibit a complete absence of zcub5 gene function, referred to as “knockout mice” (Snouwaert et al., Science 257:1083, 1992), can also be generated (Lowell et al., Nature 366:740-742, 1993). These mice can be employed to study the zcub5 gene and the protein encoded thereby in an in vivo system. Transgenic mice are particularly useful for investigating the role of zcub5 proteins in early development in that they allow the identification of developmental abnormalities or blocks resulting from the over- or underexpression of a specific factor. See also, Maisonpierre et al., Science 277:55-60, 1997 and Hanahan, Science 277:48-50, 1997. Exemplary promoters for transgenic expression include promoters from metallothionein and albumin genes.

Antisense methodology can be used to inhibit zcub5 gene transcription to examine the effects of such inhibition in vivo. Polynucleotides that are complementary to a segment of a zcub5-encoding polynucleotide (e.g., a polynucleotide as set forth in SEQ ID NO:1) are designed to bind to zcub5-encoding mRNA and to inhibit translation of such mRNA. Such antisense oligonucleotides can also be used to inhibit expression of zcub5 polypeptide-encoding genes in cell culture.

The polypeptides, nucleic acids, and antibodies of the present invention may be used in diagnosis or treatment of disorders associated with abnormal cell proliferation, including cancer, impaired or excessive vasculogenesis or angiogenesis, and diseases of the nervous system. Labeled zcub5 polypeptides may be used for imaging tumors or other sites of abnormal cell proliferation. Because angiogenesis in adult animals is generally limited to wound healing and the female reproductive cycle, it is a very specific indicator of pathological processes. Angiogenesis is associated with, for example, developing solid tumors, retinopathies (including diabetic retinopathy and macular degeneration), atherosclerosis, psoriasis, and rheumatoid arthritis. Zcub5 proteins may be useful in the treatment of these and other growth factor-dependent pathologies.

Proteins comprising the wild-type zcub5 CUB domain and variants thereof may be used to modulate activities mediated by cell-surface semaphorins. Zcub5 may thus be used to design agonists and antagonists of neuropilin-semaphorin interactions. For example, a soluble zcub5 protein may be used to inhibit semaphorin activity and thereby promote neurite outgrowth. Zcub5 proteins may thus find use in the repair of neurological damage due to strokes, head injuries, and spinal injuries, and in the treatment of neurodegenerative diseases such as multiple sclerosis, Alzheimer's disease, and Parkinson's disease. The proteins may also find use in mediating development and innervation of stomach tissue. Semaphorins have also been implicated in the development of autoimmune diseases (including rheumatoid arthritis), various forms of cancer, inflammation, retinopathies, hemangiomas, neuropathies, acute nerve damage, and ischemic events within tissues including the heart, kidney and peripheral arteries. Inhibitors of semaphorin activity are expected to find application in the treatment of these conditions.

Zcub5 polypeptides can be administered alone or in combination with other bioactive agents, such as anti-angiogenic agents or other growth factor antagonists. When using zcub5 in combination with an additional agent, the two compounds can be administered simultaneously or sequentially as appropriate for the specific condition being treated.

For pharmaceutical use, zcub5 proteins are formulated for topical or parenteral, particularly intravenous or subcutaneous, delivery according to conventional methods. In general, pharmaceutical formulations will include a zcub5 polypeptide in combination with a pharmaceutically acceptable vehicle, such as saline, buffered saline, 5% dextrose in water, or the like. Formulations may further include one or more excipients, preservatives, solubilizers, buffering agents, albumin to prevent protein loss on vial surfaces, etc. Methods of formulation are well known in the art and are disclosed, for example, in Remington: The Science and Practice of Pharmacy, Gennaro, ed., Mack Publishing Co., Easton, Pa., 19th ed., 1995. Zcub5 will generally be used in a concentration of about 10 to 100 μg/ml of total volume, although concentrations in the range of 1 ng/ml to 1000 μg/ml may be used. For topical application the protein will be applied in the range of 0.1-10 μg/cm of surface area. The exact dose will be determined by the clinician according to accepted standards, taking into account the nature and severity of the condition to be treated, patient traits, etc. Determination of dose is within the level of ordinary skill in the art. Dosing is daily or intermittently over the period of treatment. Intravenous administration will be by bolus injection or infusion over a typical period of one to several hours. Sustained release formulations can also be employed. In general, a therapeutically effective amount of zcub5 is an amount sufficient to produce a clinically significant change in the treated condition, such as a clinically significant improvement in immune system function, reduction in tumor size, or reduction in angiogenesis.

As a cell-surface molecule, zcub5 provides a means of identifying, labeling, and isolating selected cell types, and provides a target for cell-specific delivery of diagnostic and therapeutic agents. Antibodies to zcub5 or other zcub5-specific binding partners can used within known methods. For example, a labeled antibody or other binding partner can be used for in vivo or in vitro labeling of zcub5-expressing cells for a variety of purposes including, without limitation, in vivo imaging and fluorescence-activated cell sorting (FACS). Labeled antibodies or other binding partners can also be used to quantitate the levels of zcub5 polypeptides in a biological sample (e.g., blood, serum, urine) as an indicator of disease. Immobilized antibodies or other binding partners can be used to isolate or enrich for cells expressing cell-surface zcub5. Zcub5 polypeptides, anti-zcub5 antibodies, other polypeptides and proteins, and bioactive fragments or portions thereof, can be directly or indirectly coupled to detectable or cytotoxic molecules and delivered to a mammal having cells, tissues, or organs that express the anti-complementary molecule. Suitable direct tags or labels include radionuclides, enzymes, substrates, cofactors, inhibitors, fluorescent markers, chemiluminescent markers, magnetic particles and the like; indirect tags or labels may feature use of biotin-avidin or other complement/anti-complement pairs as intermediates. Suitable cytotoxic molecules include bacterial and plant toxins (for instance, diphtheria toxin, Pseudomonas exotoxin, ricin, abrin, saporin, and the like), as well as therapeutic radionuclides, such as iodine-131, rhenium-188, and yttrium-90. These can be either directly attached to the polypeptide or antibody, or indirectly attached according to known methods, such as through a chelating moiety. Polypeptides and proteins can also be conjugated to cytotoxic drugs, such as adriamycin. For indirect attachment of a detectable or cytotoxic molecule, the detectable or cytotoxic molecule may be conjugated with a member of a complementary/anticomplementary pair, where the other member is bound to the polypeptide or protein portion. For these purposes, biotin/streptavidin is an exemplary complementary/anticomplementary pair.

Polypeptide-toxin fusion proteins or antibody/fragment-toxin fusion proteins may be used for targeted cell or tissue inhibition or ablation, such as in cancer therapy. Of particular interest in this regard are conjugates of a zcub5 polypeptide and a cytotoxin, which can be used to target the cytotoxin to a tumor or other tissue that is undergoing undesired angiogenesis or neovascularization. Target cells (i.e., those displaying the zcub5 receptor) bind the zcub5-toxin conjugate, which is then internalized, killing the cell. The effects of receptor-specific cell killing (target ablation) are revealed by changes in whole animal physiology or through histological examination. Thus, ligand-dependent, receptor-directed cyotoxicity can be used to enhance understanding of the physiological significance of a protein ligand. An example of such a toxin is saporin. Manmalian cells have no receptor for saporin, which is non-toxic when it remains extracellular.

In another embodiment, zcub5-cytokine fusion proteins or antibody/fragment-cytokine fusion proteins may be used for enhancing in vitro cytotoxicity (for instance, that mediated by monoclonal antibodies against tumor targets) and for enhancing in vivo killing of target tissues (for example, blood and bone marrow cancers). See, generally, Homick et al., Blood 89:4437-4447, 1997). In general, cytokines are toxic if administered systemically. The described fusion proteins enable targeting of a cytokine to a desired site of action, such as a cell having binding sites for zcub5, thereby providing an elevated local concentration of cytokine. Suitable cytokines for this purpose include, for example, interleukin-2 and granulocyte-macrophage colony-stimulating factor (GM-CSF). Such fusion proteins may be used to cause cytokine-induced killing of tumors and other tissues undergoing angiogenesis or neovascularization.

The bioactive polypeptide or antibody conjugates described herein can be delivered intravenously, intra-arterially or intraductally, or may be introduced locally at the intended site of action.

Within the laboratory research field, zcub5 proteins can also be used as molecular weight standards or as reagents in assays for determining circulating levels of the protein, such as in the diagnosis of disorders characterized by over- or under-production of zcub5 protein or in the analysis of cell phenotype. Zcub5 proteins can be labeled and used in the study of growth factor or semaphorin biology, or immobilized and used in the purification of growth factors or semaphorins. Antibodies to zcub5 can be used in assays of zcub5 production or processing, as well as in identifying, isolating, and labeling cells as disclosed above.

Zcub5 proteins can also be used to identify inhibitors of their activity. Test compounds are added to the assays disclosed above to identify compounds that inhibit the activity of zcub5 protein. In addition to those assays disclosed above, samples can be tested for inhibition of zcub5 activity within a variety of assays designed to measure receptor binding or the stimulation/inhibition of zcub5-dependent cellular responses. For example, zcub5-responsive cell lines can be transfected with a reporter gene construct that is responsive to a zcub5-stimulated cellular pathway. Reporter gene constructs of this type are known in the art, and will generally comprise a zcub5-activated serum response element (SRE) operably linked to a gene encoding an assayable protein, such as luciferase. Candidate compounds, solutions, mixtures or extracts are tested for the ability to inhibit the activity of zcub5 on the target cells as evidenced by a decrease in zcub5 stimulation of reporter gene expression. Assays of this type will detect compounds that directly block zcub5 binding to cell-surface receptors, as well as compounds that block processes in the cellular pathway subsequent to receptor-ligand binding. In the alternative, compounds or other samples can be tested for direct blocking of zcub5 binding to receptor using zcub5 tagged with a detectable label (e.g., ¹²⁵I, biotin, horseradish peroxidase, FITC, and the like). Within assays of this type, the ability of a test sample to inhibit the binding of labeled zcub5 to the receptor is indicative of inhibitory activity, which can be confirmed through secondary assays. Receptors used within binding assays may be cellular receptors or isolated, immobilized receptors.

As used herein, the term “antibodies” includes polyclonal antibodies, monoclonal antibodies, antigen-binding fragments thereof such as F(ab′)₂ and Fab fragments, single chain antibodies, and the like, including genetically engineered antibodies. Non-human antibodies may be humanized by grafting non-human CDRs onto human framework and constant regions, or by incorporating the entire non-human variable domains (optionally “cloaking” them with a human-like surface by replacement of exposed residues, wherein the result is a “veneered” antibody). In some instances, humanized antibodies may retain non-human residues within the human variable region framework domains to enhance proper binding characteristics. Through humanizing antibodies, biological half-life may be increased, and the potential for adverse immune reactions upon administration to humans is reduced. One skilled in the art can generate humanized antibodies with specific and different constant domains (i.e., different Ig subclasses) to facilitate or inhibit various immune functions associated with particular antibody constant domains. Antibodies are defined to be specifically binding if they bind to a zcub5 polypeptide or protein with an affinity at least 10-fold greater than the binding affinity to control (non-zcub5) polypeptide or protein. The affinity of a monoclonal antibody can be readily determined by one of ordinary skill in the art (see, for example, Scatchard, Ann. NY Acad. Sci. 51: 660-672, 1949).

Methods for preparing polyclonal and monoclonal antibodies are well known in the art (see for example, Hurrell, J. G. R., Ed., Monoclonal Hybridoma Antibodies: Techniques and Applications, CRC Press, Inc., Boca Raton, Fla., 1982). As would be evident to one of ordinary skill in the art, polyclonal antibodies can be generated from a variety of warm-blooded animals such as horses, cows, goats, sheep, dogs, chickens, rabbits, mice, and rats. The immunogenicity of a zcub5 polypeptide may be increased through the use of an adjuvant such as alum (aluminum hydroxide) or Freund's complete or incomplete adjuvant. Polypeptides useful for immunization also include fusion polypeptides, such as fusions of a zcub5 polypeptide or a portion thereof with an immunoglobulin polypeptide or with maltose binding protein. If the zcub5 polypeptide portion is “hapten-like”, such portion may be advantageously joined or linked to a macromolecular carrier (such as keyhole limpet hemocyanin (KLH), bovine serum albumin (BSA) or tetanus toxoid) for immunization.

Alternative techniques for generating or selecting antibodies include in vitro exposure of lymphocytes to zcub5 polypeptides, and selection of antibody display libraries in phage or similar vectors (e.g., through the use of immobilized or labeled zcub5 polypeptide). Human antibodies can be produced in transgenic, non-human animals that have been engineered to contain human immunoglobulin genes as disclosed in WIPO Publication WO 98/24893. It is preferred that the endogenous immunoglobulin genes in these animals be inactivated or eliminated, such as by homologous recombination.

A variety of assays known to those skilled in the art can be utilized to detect antibodies that specifically bind to zcub5 polypeptides. Exemplary assays are described in detail in Antibodies: A Laboratory Manual, Harlow and Lane (Eds.), Cold Spring Harbor Laboratory Press, 1988. Representative examples of such assays include concurrent immunoelectrophoresis, radio-immunoassays, radio-immunoprecipitations, enzyme-linked immunosorbent assays (ELISA), dot blot assays, Western blot assays, inhibition or competition assays, and sandwich assays.

Antibodies to zcub5 may be used for affinity purification of the protein; within diagnostic assays for determining circulating levels of the protein; for detecting or quantitating soluble zcub5 polypeptide as a marker of underlying pathology or disease; for immunolocalization within whole animals or tissue sections, including immunodiagnostic applications; for immunohistochemistry; and as antagonists to block protein activity in vitro and in vivo. Antibodies to zcub5 may also be used for tagging cells that express zcub5; for affinity purification of zcub5 polypeptides and proteins; in analytical methods employing FACS; for screening expression libraries; and for generating anti-idiotypic antibodies. Antibodies can be linked to other compounds, including therapeutic and diagnostic agents, using known methods to provide for targetting of those compounds to cells expressing receptors for zcub5. For certain applications, including in vitro and in vivo diagnostic uses, it is advantageous to employ labeled antibodies. Antibodies of the present invention may also be directly or indirectly conjugated to drugs, toxins, radionuclides and the like, and these conjugates used for in vivo diagnostic or therapeutic applications. See, in general, Ramakrishnan et al., Cancer Res. 56:1324-1330, 1996. For in vivo use, an anti-zcub5 antibody or other binding partner can be directly or indirectly coupled to a detectable molecule and delivered to a mammal having cells, tissues, or organs that express zcub5. Suitable detectable molecules include radionuclides, enzymes, substrates, cofactors, inhibitors, fluorescent markers, chemiluminescent markers, magnetic particles, electron-dense compounds, heavy metals, and the like. These can be either directly attached to the antibody or other binding partner, or indirectly attached according to known methods, such as through a chelating moiety. For indirect attachment of a detectable molecule, the detectable molecule can be conjugated with a first member of a complementary/anticomplementary pair, wherein the second member of the pair is bound to the anti-zcub5 antibody or other binding partner. Biotin/streptavidin is an exemplary complementary/anticomplementary pair; others will be evident to those skilled in the art. The labeled compounds described herein can be delivered intravenously, intra-arterially or intraductally, or may be introduced locally at the intended site of action.

Antibodies to zcub5 polypeptides may be used therapeutically where it is desirable to inhibit the activity of zcub5, for example by blocking the binding of a zcub 5 polypeptide to a member of the PDGF/VEGF family. The antibodies may thus be used to effectively increase the level of PDGF/VEGF activity in a patient, particularly under circumstances wherein zcub5 expression is abnormally elevated.

The present invention also provides reagents for use in diagnostic applications. For example, the zcub5 gene, a probe comprising zcub5 DNA or RNA, or a subsequence thereof can be used to determine the presence of mutations at or near the zcub5 locus at human chromosome 6q21. This region of chromosome 6 has been associated with retinitis pigmentosa. See, OMIM™ Database, Johns Hopkins University, 2000 (http://www.ncbi.nlm.nih.gov/entrez/query.fcgi?db=OMIM).

Detectable chromosomal aberrations at the zcub5 gene locus include, but are not limited to, aneuploidy, gene copy number changes, insertions, deletions, restriction site changes, and rearrangements. These aberrations can occur within the coding sequence, within introns, or within flanking sequences, including upstream promoter and regulatory regions, and may be manifested as physical alterations within a coding sequence or changes in gene expression level. Analytical probes will generally be at least 20 nucleotides in length, although somewhat shorter probes (14-17 nucleotides) can be used. PCR primers are at least 5 nucleotides in length, usually 15 or more nt, and commonly 20-30 nt. Short polynucleotides can be used when a small region of the gene is targetted for analysis. For gross analysis of genes, a polynucleotide probe may comprise an entire exon or more. Probes will generally comprise a polynucleotide linked to a signal-generating moiety such as a radionucleotide. In general, these diagnostic methods comprise the steps of (a) obtaining a genetic sample from a patient; (b) incubating the genetic sample with a polynucleotide probe. or primer as disclosed above, under conditions wherein the polynucleotide will hybridize to complementary polynucleotide sequence, to produce a first reaction product; and (c) comparing the first reaction product to a control reaction product. A difference between the first reaction product and the control reaction product is indicative of a genetic abnormality in the patient. Genetic samples for use within the present invention include genornic DNA, cDNA, and RNA. The polynucleotide probe or primer can be RNA or DNA, and will comprise a portion of SEQ ID NO:1, the complement of SEQ ID NO:1, or an RNA equivalent thereof. Suitable assay methods in this regard include molecular genetic techniques known to those in the art, such as restriction fragment length polymorphism (RFLP) analysis, short tandem repeat (STR) analysis employing PCR techniques, ligation chain reaction (Barany, PCR Methods and Applications 1:5-16, 1991), ribonuclease protection assays, and other genetic linkage analysis techniques known in the art (Sambrook et al., ibid.; Ausubel et. al., ibid.; A. J. Marian, Chest 108:255-265, 1995). Ribonuclease protection assays (see, e.g., Ausubel et al., ibid., ch. 4) comprise the hybridization of an RNA probe to a patient RNA sample, after which the reaction product (RNA-RNA hybrid) is exposed to RNase. Hybridized regions of the RNA are protected from digestion. Within PCR assays, a patient genetic sample is incubated with a pair of polynucleotide primers, and the region between the primers is amplified and recovered. Changes in size or amount of recovered product are indicative of mutations in the patient. Another PCR-based technique that can be employed is single strand conformational polymorphism (SSCP) analysis (Hayashi, PCR Methods and Applications 1:34-38, 1991).

Radiation hybrid mapping is a somatic cell genetic technique developed for constructing high-resolution, contiguous maps of mammalian chromosomes (Cox et al., Science 250:245-250, 1990). Partial or full knowledge of a gene's sequence allows one to design PCR primers suitable for use with chromosomal radiation hybrid mapping panels. Radiation hybrid mapping panels that cover the entire human genome are commercially available, such as the Stanford G3 RH Panel and the GeneBridge 4 RH Panel (Research Genetics, Inc., Huntsville, Ala.). These panels enable rapid, PCR-based chromosomal localizations and ordering of genes, sequence-tagged sites (STSs), and other nonpolymorphic and polymorphic markers within a region of interest, and the establishment of directly proportional physical distances between newly discovered genes of interest and previously mapped markers. The precise knowledge of a gene's position can be useful for a number of purposes, including: 1) determining if a sequence is part of an existing contig and obtaining additional surrounding genetic sequences in various forms, such as YACs, BACs or cDNA clones; 2) providing a possible candidate gene for an inheritable disease which shows linkage to the same chromosomal region; and 3) cross-referencing model organisms, such as mouse, which may aid in determining what function a particular gene might have.

Sequence tagged sites (STSs) can also be used independently for chromosomal localization. An STS is a DNA sequence that is unique in the human genome and can be used as a reference point for a particular chromosome or region of a chromosome. An STS is defined by a pair of oligonucleotide primers that are used in a polymerase chain reaction to specifically detect this site in the presence of all other genomic sequences. Since STSs are based solely on DNA sequence they can be completely described within an electronic database, for example, Database of Sequence Tagged Sites (dbSTS), GenBank (National Center for Biological Information, National Institutes of Health, Bethesda, MD http://www.ncbi.nlm.nih.gov), and can be searched with a gene sequence of interest for the mapping data contained within these short genomic landmark STS sequences.

Inhibitors of cell-surface zcub5 activity include anti-zcub5 antibodies and soluble zcub5 proteins, as well as other peptidic and non-peptidic agents (including inhibitory polynucleotides). Such antagonists can be used to block the effects of zcub5 ligands (e.g., semaphorins or members of the PDGF/VEGF family) on cells or tissues. Of particular interest is the use of such antagonists in reducing angiogenesis, reducing cancer growth and metastasis, and modulating immune system functions. Antagonists are formulated for pharmaceutical use as generally disclosed above, taking into account the precise chemical and physical nature of the inhibitor and the condition to be treated.

The relevant determinations are within the level of ordinary skill in the formulation art.

Polynucleotides encoding zcub5 polypeptides are useful within gene therapy applications where it is desired to increase or inhibit zcub5 activity. If a mammal has a mutated or absent zcub5 gene, a zcub5 gene can be introduced into the cells of the mammal. In one embodiment, a gene encoding a zcub5 polypeptide is introduced in vivo in a viral vector. Such vectors include an attenuated or defective DNA virus, such as, but not limited to, herpes simplex virus (HSV), papillomavirus, Epstein Barr virus (EBV), adenovirus, adeno-associated virus (AAV), and the like. Defective viruses, which entirely or almost entirely lack viral genes, are preferred. A defective virus is not infective after introduction into a cell. Use of defective viral vectors allows for administration to cells in a specific, localized area, without concern that the vector can infect other cells. Examples of particular vectors include, but are not limited to, a defective herpes simplex virus 1 (HSV1) vector (Kaplitt et al., Molec. Cell. Neurosci. 2:320-330, 1991); an attenuated adenovirus vector, such as the vector described by Stratford-Perricaudet et al., J. Clin. Invest. 90:626-630, 1992; and a defective adeno-associated virus vector (Samulski et al., J. Virol. 61:3096-3101, 1987; Samulski et al., J. Virol. 63:3822-3888, 1989). Within another embodiment, a zcub5 gene can be introduced in a retroviral vector as described, for example, by Anderson et al., U.S. Pat. No. 5,399,346; Mann et al. Cell 33:153, 1983; Temin et al., U.S. Pat. No. 4,650,764; Temin et al., U.S. Pat. No. 4,980,289; Markowitz et al., J. Virol. 62:1120, 1988; Temin et al., U.S. Pat. No. 5,124,263; Dougherty et al., WIPO Publication WO 95/07358; and Kuo et al., Blood 82:845, 1993. Alternatively, the vector can be introduced by liposome-mediated transfection (“lipofection”). Synthetic cationic lipids can be used to prepare liposomes for in vivo transfection (Felgner et al., Proc. Natl. Acad. Sci. USA 84:7413-7417, 1987; Mackey et al., Proc. Natl. Acad. Sci. USA 85:8027-8031, 1988). The use of lipofection to introduce exogenous genes into specific organs in vivo has certain practical advantages, including molecular targeting of liposomes to specific cells. Directing transfection to particular cell types is particularly advantageous in a tissue with cellular heterogeneity, such as the pancreas, liver, kidney, and brain. Lipids may be chemically coupled to other molecules for the purpose of targeting. Peptidic and non-peptidic molecules can be coupled to liposomes chemically. Within another embodiment, cells are removed from the body, a vector is introduced into the cells as a naked DNA plasmid, and the transformed cells are re-implanted into the body as disclosed above.

Inhibitory polynucleotides can be used to inhibit zcub5 gene transcription or translation in a patient. Polynucleotides that are complementary to a segment of a zcub5-encoding polvnucleotide (e.g., a polynucleotide as set forth in SEQ ID NO:1) are designed to bind to zcub5-encoding mRNA and to inhibit translation of such mRNA. Such antisense polynucleotides can be targetted to specific tissues using a gene therapy approach with specific vectors and/or promoters, such as viral delivery systems. Ribozymes can also be used as zcub5 antagonists. Ribozymes are RNA molecules that contains a catalytic center and a target RNA binding portion. The term includes RNA enzymes, self-splicing RNAs, self-cleaving RNAs, and nucleic acid molecules that perform these catalytic functions. A ribozyme selectively binds to a target RNA molecule through complementary base pairing, bringing the catalytic center into close proximity with the target sequence. The ribozyme then cleaves the target RNA and is released, after which it is able to bind and cleave additional molecules. A nucleic acid molecule that encodes a ribozyme is termed a “ribozyme gene.” Ribozymes can be designed to express endonuclease activity that is directed to a certain target sequence in a mRNA molecule (see, for example, Draper and Macejak, U.S. Pat. No. 5,496,698, McSwiggen, U.S. Pat. No. 5,525,468, Chowrira and McSwiggen, U.S. Pat. No. 5,631,359, and Robertson and Goldberg, U.S. Pat. No. 5,225,337). An expression vector can be constructed in which a regulatory element is operably linked to a nucleotide sequence that encodes a ribozyme. In another approach, expression vectors can be constructed in which a regulatory element directs the production of RNA transcripts capable of promoting RNase P-mediated cleavage of mRNA molecules that encode a zcub5 polypeptide. According to this approach, an external guide sequence can be constructed for directing the endogenous ribozyme, RNase P, to a particular species of intracellular mRNA, which is subsequently cleaved by the cellular ribozyme (see, for example, Altman et al., U.S. Pat. No. 5,168,053; Yuan et al., Science 263:1269, 1994; Pace et al., WIPO Publication No. WO 96/18733; George et al., WIPO Publication No. WO 96/21731; and Werner et al., WIPO Publication No. WO 97/33991). An external guide sequence generally comprises a ten- to fifteen-nucleotide sequence complementary to zcub5 MRNA, and a 3′-NCCA nucleotide sequence, wherein N is preferably a purine. The external guide sequence transcripts bind to the targeted mRNA species by the formation of base pairs between the mRNA and the complementary external guide sequences, thus promoting cleavage of MRNA by RNase P at the nucleotide located at the 5′-side of the base-paired region.

Polynucleotides and polypeptides of the present invention will additionally find use as educational tools within laboratory practicum kits for courses related to genetics, molecular biology, protein chemistry, and antibody production and analysis. Due to their unique polynucleotide and polypeptide sequences, molecules of zcub5 can be used as standards or as “unknowns” for testing purposes. For example, zcub5 polynucleotides can be used as an aid in teaching a student how to prepare expression constructs for bacterial, viral, and/or mammalian expression, including fusion constructs, wherein zcub5 is the gene to be expressed; for determining the restriction endonuclease cleavage sites of the polynucleotides (see Table 3); determining mRNA and DNA localization of zcub5 polynucleotides in tissues (e.g., by Northern blotting, Southern blotting, or polymerase chain reaction); and for identifying related polynucleotides and polypeptides by nucleic acid hybridization.

TABLE 3

| Enzyme | Cut Site(s) | Enzyme | Cut Site(s) |
|---|---|---|---|
| ApaI | 1953 | FspI | 1926, 3120 |
| BamHI | 27, 479 | HpaI | 2878 |
| BbsI | 1148, 581 | KasI | 1855, 86, 2012, 61 |
| BglI | 59, 1775 | MscI | 720, 773, 939, 1241 |
| BglII | 1666 | NaeI | 1828, 1798 |
| BpmI | 1037 | NarI | 1856, 2013, 62, 87 |
| BsaI | 936, 157, 909 | NcoI | 1755 |
| BseRI | 194, 1787, 1969 | NgoMI | 1826, 1796 |
| BsmBI | 606 | NheI | 560 |
| Bsp120I | 1949 | PflMI | 410 |
| BspHI | 1707, 2618 | PmlI | 1900 |
| BsrBI | 1894, 1932, 163 | PpuMI | 255, 1204 |
| BssHII | 1906 | PshAI | 1609 |
| BstEII | 2517 | PstI | 174 |
| BstXI | 2529, 1011, 591 | PvuII | 1616, 602, 2458, 16, |
| | | | 2831 |
| ClaI | 1023 | SacI | 1302 |
| DraI | 2427 | SacII | 94 |
| DraIII | 2231, 208 | SapI | 391 |
| EagI | 1828 | SmaI | 2146, 253, 10 |
| EarI | 2492, 391, 380 | SrfI | 10 |
| Ec1136II | 1300 | SspI | 2591 |
| Eco57I | 372, 719, 935, 2466, 2985, | Tth111I | 2715 |
| | 2613 | | |
| EcoNI | 2793 | XcmI | 1885, 73 |
| EcoRV | 340 | XmaI | 2144, 8, 251 |
| FseI | 1830 | XmnI | 765, 2690 |

Zcub5 polypeptides can be used educationally as aids in teaching preparation of antibodies; identification of proteins by Western blotting; protein purification; determination of the weight of expressed zcub5 polypeptides as a ratio to total protein expressed; identification of peptide cleavage sites (see FIGS. 3A-3F); coupling of amino and carboxyl terminal tags; amino acid sequence analysis; as well as, but not limited to, monitoring biological activities of both the native and tagged protein (i.e., receptor binding, signal transduction, proliferation, and differentiation) in vitro and in vivo. Zcub5 polypeptides can also be used to teach analytical skills such as mass spectrometry, circular dichroism to determine conformation, in particular the locations of the disulfide bonds, x-ray crystallography to determine the three-dimensional structure in atomic detail, nuclear magnetic resonance spectroscopy to reveal the structure of proteins in solution, and the like. For example, a kit containing a zcub5 polypeptide can be given to a student to analyze. Since the amino acid sequence would be known by the instructor, the protein can be given to the student as a test to determine the skills or develop the skills of the student, and the instructor would then know whether or not the student had correctly analyzed the polypeptide. Since every polypeptide is unique, the educational utility of zcub5 would be unique unto itself.

The antibodies which bind specifically to zcub5 can be used as a teaching aid to instruct students how to prepare affinity chromatography columns to purify zcub5, cloning and sequencing the polynucleotide that encodes an antibody and thus as a practicum for teaching a student how to design humanized antibodies. The zcub5 gene, polypeptide or antibody would then be packaged by reagent companies and sold to universities so that the students gain skill in art of molecular biology. Because each gene and protein is unique, each gene and protein creates unique challenges and learning experiences for students in a lab practicum. Such educational kits containing the zcub5 gene, polypeptide, or antibody are considered within the scope of the present invention.

The invention is further illustrated by the following non-limiting examples.

### EXAMPLES

### Example 1

A PCR panel comprising 94 cDNA samples plus human genomic DNA and water controls was screened for the presence of zcub5 sequences. PCR reactions were set up using oligonucleotide primers ZC28,499 (SEQ ID NO:18) and ZC28,500 (SEQ ID NO:19), a DNA polymerase (Ex Taq™; Takara Shozu, Japan) plus antibody mixture, an annealing temperature of 63.0° C., and an extension time of 30 seconds for a total of 35 cycles. Results are shown in Table 4.

TABLE 4

| | Re- | | Re- |
|---|---|---|---|
| Source* | sult | Source | sult |
| Adrenal Gland | − | Bladder | − |
| Bone Marrow | − | Brain | − |
| Cervix | + | Colon | + |
| Fetal Brain | − | Fetal Heart | +/− |
| Fetal Kidney | − | Fetal Liver | + |
| Fetal Lung | − | Fetal muscle | − |
| Fetal Skin | − | Heart | − |
| Heart | − | K562 (human chronic | − |
| | | myelogenous leukemia) | |
| Kidney | − | Liver | − |
| Lung | − | Lymph Node | − |
| Melanoma | − | Pancreas | − |
| Pituitary | +/− | Placenta | + |
| Prostate | − | Rectum | + |
| Salivary Gland | − | Skeletal Muscle | − |
| Small Intestine | + | Spinal Cord | − |
| Spleen | + | Stomach | +/− |
| Testis | − | Testis | − |
| Thymus | − | Thyroid | − |
| Trachea | − | Uterus | − |
| bone marrow library | − | fetal brain library | + |
| islet library | − | prostate 0.5-1.6 kb library | − |
| prostate >1.6 kb library | + | prostate smc library | − |
| RPMI 1788 (B−cell) library | + | testis library | + |
| thyroid Library | + | WI38 (lung fibroblast) | + |
| | | library | |
| WI−38 (lung fibroblast) | + | RPMI 1788 (B-cell) library | + |
| library | | | |
| Thyroid library | + | Islet library | + |
| HPV (prostate epithelia) | + | HaCAT (keratinocyte) library | + |
| library | | | |
| Bone Marrow library | − | Adrenal Gland library | + |
| Prostate smc library | + | Prostate library | − |
| Fetal Brain library | + | Testis library | + |
| HPVS (prostate epithelia) | + | CD3+ (2) library | − |
| library | | | |
| Fetal Brain arr. library | + | Bone Marrow arr. library | + |
| Pituitary arr. library | − | Heart arr. library | − |
| Salivary Gland arr. library | − | Placenta arr. library | − |
| Testis 10 K pools arr. | + | Testis 1 K pools arr. library | + |
| library | | | |
| Gastric CA | − | Esophagus CA | + |
| Liver CA | − | Kidney CA | + |
| Ovarian CA | + | Lung CA | + |
| Uterus CA | − | Rectal CA | + |
| Platelet library | − | HL60 (promyelocytic | − |
| | | leukemia) | |
| HL60 λgt11 library | − | HBL−100 (breast epithelial) | − |
| | | library | |
| Renal mesangial library | +/− | HL60 #2 λgt11 | − |
| #2 λgt11 | | | |
| Neutrophil λgt11 library | − | T-cell λgt11 library | − |
| | | 3.83 × 10⁷ pfu | |
| Fibroblast λgt11 library | + | Entamoeba histolytica λgt11 | − |
| Endothelial λgt11 library | − | HepG2 (hepatoma) λgt11 | − |
| HUT-102 (T-cell | − | gt 11 placenta cDNA library | − |
| lymphoma) cDNA | | | |
| lysate 5.17 × 10⁷ pfu/μL | | | |
| Genomic DNA | n.d. | MPC λgt11 | − |
| | | Water | − |

*Abbreviations used: CA, cancer; SMC, smooth muscle cell; arr., arrayed.

### Example 2

PCR products (˜217 bp fragments) from the HPVS, testis, and ovarian cancer samples (Example 1) were sequenced. The HPVS fragment was confirmed to be a human zcub5 sequence. DNA fragments comprising zcub5 sequences were also obtained from various public and private sources and sequenced. A full-length zcub5 DNA was constructed by digestion and ligation of three clones from various sources. A consensus human zcub5 sequence is shown in SEQ ID NO:1, and the encoded amino acid sequence is shown in SEQ ID NO:2.

### Example 3

A mouse cDNA panel was screened for zcub5 by PCR using oligonucleotide primers ZC 28,497 (SEQ ID NO:16) and ZC28,498 (SEQ ID NO:17), and a DNA polymerase (Ex Taq™; Takara Shozu, Japan) plus antibody mixture. The reaction was run for 35 cycles at an annealing temperature of 64. 1° C. with an extension time of 30 seconds. Positive results were obtained from brain, 15-day embryo library total pool, kidney, pancreas, salivary gland, spleen, stomach, testis, uterus, liver, lung, skeletal muscle, 7-day embryo, 11-day embryo, 15-day embryo, and 17-day embryo. Fragments (˜145 bp) from 15-day embryo library total pool, 15-day embryo, and kidney were sequenced and confirmed to be zcub5 sequence.

The mouse 15-day embryo library was chosen for further screening. This library was an arrayed library representing 9.6×10⁵ clones in the pCMV sport2 vector. A working plate containing 80 pools of 12,000 colonies each was screened by PCR for the presence of zcub5 DNA using oligonucleotide primers ZC28,497 (SEQ ID NO:16) and ZC28,498 (SEQ ID NO:17) with an annealing temperature of 64.1° C. for 35 cycles.

One positive pool was plated and transferred to nylon filters (Hybond-NTM; Amersham Corporation, UK). The filters were denatured for 6 minutes in 0.5 M NaOH and 1.5 M Tris-HCl, pH 7.2, then neutralized in 1.5 M NaCl and 0.5 M Tris-HCl, pH 7.2 for 6 minutes. The DNA was affixed to the filters using a UV crosslinker (Stratalinker®; Stratagene, La Jolla, Calif.) at 1200 joules. The filters were prewashed at 65° C. in prewash buffer consisting of 0.25× SSC, 0.25% SDS, and 1 mM EDTA. The solution was changed a total of three times over a 45-minute period to remove cell debris. Filters were prehybridized overnight at 65° C. in 20 ml of a commercially available hybridization solution (ExpressHyb™ Hybridization Solution; Clontech Laboratories, Inc., Palo Alto, Calif.). A probe was generated by PCR using oligonucleotide primers ZC28,497 (SEQ ID NO:16) and ZC28,498 (SEQ ID NO:17), and an annealing temperature of 64.1° C. for 35 cycles and a mouse 15-day embryo cDNA library as template. The resulting PCR fragment was gel purified using a spin column containing a silica gel membrane (QIAquick™ Gel Extraction Kit; Qiagen, Inc., Valencia, Calif.). The probe was radioactively labeled with ³²p using a commercially available kit (Rediprime™ II random-prime labeling system; Amersham Corp., UK) according to the manufacturer's specifications and purified using a commercially available push column (NucTrap® column; Stratagene, La Jolla, Calif.). Hybridization took place overnight at 65° C. in commercially available hybridization solution. Filters were rinsed 6 times at 65° C. in pre-wash buffer, then exposed to film overnight at −80° C. Individual positive colonies were screened by PCR using oligonucleotide primers ZC28,497 (SEQ ID NO:16) and ZC28,498 (SEQ ID NO:17) and an annealing temp of 61.0° C. Two positive clones were sequenced and found to contain the same, full-length zcub5 sequence (SEQ ID NO:5), which was designated “muzcub5×3finalseq.”

Two expressed sequence tags (EST) corresponding to portions of the zcub5 sequence were identified in a public database. Clones comprising these sequences were sequenced and found to encode an apparent splice variant of mouse zcub5 as shown in SEQ ID NO:3 (designated “muzcub5×2finalseq”).

An alignment of human and mouse zcub5 amino acid sequences is shown in FIG. 2. The gaps within the mouse sequences are believed to be due to alternative splicing.

### Example 4

Recombinant zcub5 is produced in E. coli using a His₆ tag/maltose binding protein (MBP) double affinity fusion system as generally disclosed by Pryor and Leiting, Prot. Expr. Pur. 10:309-319, 1997. A thrombin cleavage site is placed at the junction between the affinity tag and zcub5 sequences.

The fusion construct is assembled in the vector pTAP98, which comprises sequences for replication and selection in E. coli and yeast, the E. coli tac promoter, and a unique Smal site just downstream of the MBP-His₆-thrombin site coding sequences. The zcub5 cDNA (SEQ ID NO:1) is amplified by PCR using primers each comprising 40 bp of sequence homologous to vector sequence and 25 bp of sequence that anneals to the cDNA. The reaction is run using Taq DNA polymerase (Boehringer Mannheim, Indianapolis, Ind.) for 30 cycles of 94° C., 30 seconds; 60° C., 60 seconds; and 72° C., 60 seconds. One microgram of the resulting fragment is mixed with 100 ng of Smal-cut pTAP98, and the mixture is transformed into yeast to assemble the vector by homologous recombination (Oldenburg et al., Nucl. Acids. Res. 25:451-452, 1997). Ura⁺ transformants are selected.

Plasmid DNA is prepared from yeast transformants and transformed into E. coli MC1061. Pooled plasmid DNA is then prepared from the MC1061 transformants by the rniniprep method after scraping an entire plate. Plasmid DNA is analyzed by restriction digestion.

E. coli strain BL21 is used for expression of zcub5. Cells are transformed by electroporation and grown on minimal glucose plates containing casamino acids and ampicillin.

Protein expression is analyzed by gel electrophoresis. Cells are grown in liquid glucose media containing casamino acids and ampicillin. After one hour at 37° C., IPTG is added to a final concentration of 1 mM, and the cells are grown for an additional 2-3 hours at 37° C. Cells are disrupted using glass beads, and extracts are prepared.

### Example 5

Larger scale cultures of zcub5 transformants are prepared by the method of Pryor and Leiting (ibid.). 100-ml cultures in minimal glucose media containing casamino acids and 100 μg/ml ampicillin are grown at 37° C. in 500-ml baffled flasks to OD₆₀₀=0.5. Cells are harvested by centrifugation and resuspended in 100 ml of the same media at room temperature. After 15 minutes, IPTG is added to 0.5 mM, and cultures are incubated at room temperature (ca. 22.5° C.) for 16 to 20 hours with shaking at 125 rpm. The culture is harvested by centrifugation, and cell pellets are stored at −70° C.

### Example 6

For larger-scale protein preparation, 500-ml cultures of E. coli BL21 expressing the zcub5-MBP-His₆ fusion protein are prepared essentially as disclosed in Example 5. Cell pellets are resuspended in 100 ml of Talon binding buffer (20 mM Tris, pH 7.58, 100 mM NaCl, 20 mM NaH₂PO₄, 0.4 mM 4-(2-Aminoethyl)-benzenesulfonyl fluoride hydrochloride [Pefabloc® SC; Boehringer-Mannheim], 2 35 μg/ml Leupeptin, 2 μg/ml Aprotinin). The cells are lysed in a French press at 30,000 psi, and the lysate is centrifuged at 18,000× g for 45 minutes at 4° C. to clarify it. Protein concentration is estimated by gel electrophoresis with a BSA standard.

Recombinant zcub5 fusion protein is purified from the lysate by affinity chromatography. Talon resin is equilibrated in binding buffer. One ml of packed resin per 50 mg protein is combined with the clarified supernatant in a tube, and the tube is capped and sealed, then placed on a rocker overnight at 4° C. The resin is then pelleted by centrifugation at 4° C. and washed three times with binding buffer. Protein is eluted with binding buffer containing 0.2M imidazole. The resin and elution buffer are mixed for at least one hour at 4° C., the resin is pelleted, and the supernatant is removed. An aliquot is analyzed by gel electrophoresis, and concentration is estimated. Amylose resin is equilibrated in amylose binding buffer (20 mM Tris-HCl, pH 7.0, 100 mM NaCl, 10 mM EDTA) and combined with the supernatant from the Talon resin at a ratio of 2 mg fusion protein per ml of resin. Binding and washing steps are carried out as disclosed above. Protein is eluted with amylose binding buffer containing 10 mM maltose using as small a volume as possible to minimize the need for subsequent concentration. The eluted protein is analyzed by gel electrophoresis and staining with Coomassie blue using a BSA standard, and by Western blotting using an anti-MBP antibody.

### Example 7

An expression plasmid containing all or part of a polynucleotide encoding zcub5 is constructed via homologous recombination. A fragment of zcub5 cDNA is isolated by PCR using primers that comprise, from 5′ to 3′ end, 40 bp of flanking sequence from the vector and 17 bp corresponding to the amino and carboxyl termini from the open reading frame of zcub5. The resulting PCR product includes flanking regions at the 5′ and 3′ ends corresponding to the vector sequences flanking the zcub5 insertion point. Ten μl of the 100 μl PCR reaction mixture is run on a 0.8% low-melting-temperature agarose (SeaPlaque GTG®; FMC BioProducts, Rockland, ME) gel with 1×TBE buffer for analysis. The remaining 90 μl of the reaction mixture is precipitated with the addition of 5 μl 1 M NaCl and 250 μl of absolute ethanol.

The plasmid pZMP6, which has been cut with Smal, is used for recombination with the PCR fragment. Plamid pZMP6 is a mammalian expression vector containing an expression cassette having the cytomegalovirus immediate early promoter, multiple restriction sites for insertion of coding sequences, a stop codon, and a human growth hormone terminator; an E. Coli origin of replication; a mammalian selectable marker expression unit comprising an SV40 promoter, enhancer and origin of replication, a DHER gene, and the SV40 terminator; and URA3 and CEN-ARS sequences required for selection and replication in S. cerevisiae. It was constructed from pZP9 (deposited at the American Type Culture Collection, 10801 University Boulevard, Manassas, Va. 20110-2209, under Accession No. 98668) with the yeast genetic elements taken from pRS316 (deposited at the American Type Culture Collection, 10801 University Boulevard, Manassas, Va. 20110-2209, under Accession No. 77145), an internal ribosome entry site (IRES) element from poliovirus, and the extracellular domain of CD8 truncated at the C-terminal end of the transmembrane domain.

One hundred microliters of competent yeast (S. cerevisiae) cells are combined with 10 μl of the DNA preparations from above and transferred to a 0.2-cm electroporation cuvette. The yeast/DNA mixture is electropulsed using power supply (BioRad Laboratories, Hercules, Calif.) settings of 0.75 kV (5 kV/cm), ¢ ohms, 25 μF. To each cuvette is added 600 μl of 1.2 M sorbitol, and the yeast is plated in two 300-μl aliquots onto two URA-D (selective media lacking uracil and containing glucose) plates and incubated at 30° C. After about 48 hours, the Ura⁺ yeast transformants from a single plate are resuspended in 1 ml H₂O and spun briefly to pellet the yeast cells. The cell pellet is resuspended in 1 ml of lysis buffer (2% Triton X-100, 1% SDS, 100 mM NaCl, 10 mM Tris, pH 8.0, 1 mM EDTA). Five hundred microliters of the lysis mixture is added to an Eppendorf tube containing 300 μl acid-washed glass beads and 200 11 phenol-chloroform, vortexed for 1 minute intervals two or three times, and spun for 5 minutes in an Eppendorf centrifuge at maximum speed. Three hundred microliters of the aqueous phase is transferred to a fresh tube, and the DNA is precipitated with 600 μl ethanol (EtOH), followed by centrifugation for 10 minutes at 4° C. The DNA pellet is resuspended in 10 μl H₂O.

Transformation of electrocompetent E. coli host cells (Electromax DH10B™ cells; obtained from Life Technologies, Inc., Gaithersburg, Md.) is done with 0.5-2 ml yeast DNA prep and 40 μl of cells. The cells are electropulsed at 1.7 kV, 25 μF, and 400 ohms. Following electroporation, 1 ml SOC (2% Bacto™ Tryptone (Difco, Detroit, Mich.), 0.5% yeast extract (Difco), 10 mM NaCl, 2.5 mM KCl, 10 mM MgCl₂, 10 mM MgSO₄, 20 mM glucose) is plated in 250-μl aliquots on four LB AMP plates (LB broth (Lennox), 1.8% Bacto™ Agar (Difco), 100 mg/L Ampicillin).

Individual clones harboring the correct expression construct for zcub5 are identified by restriction digest to verify the presence of the zcub5 insert and to confirm that the various DNA sequences have been joined correctly to one another.

The inserts of positive clones are subjected to sequence analysis. Larger scale plasmid DNA is isolated using a commercially available kit (QIAGEN Plasmid Maxi Kit, Qiagen, Valencia, Calif.) according to manufacturer's instructions. The correct construct is designated pZMP6/zcub5.

### Example 8

CHO DG44 cells (Chasin et al., Som. Cell. Molec. Genet. 12:555-666, 1986) are plated in 10-cm tissue culture dishes and allowed to grow to approximately 50% to 70% confluency overnight at 37° C., 5% CO₂, in Ham's F12/FBS media (Ham's F12 medium (Life Technologies), 5% fetal bovine serum (Hyclone, Logan, Utah), 1% L-glutamine (JRH Biosciences, Lenexa, Kans.), 1% sodium pyruvate (Life Technologies)). The cells are then transfected with the plasmid zcub5/pZMP6 by liposome-mediated transfection using a 3:1 (w/w) liposome formulation of the polycationic lipid 2,3-dioleyloxy-N-[2(sperminecarboxamido)ethyl]-N,N-dimethyl-1-propaniminium-trifluoroacetate and the neutral lipid dioleoyl phosphatidylethanolamine in membrane-filetered water (Lipofectamine™ Reagent, Life Technologies), in serum free (SF) media formulation (Ham's F12, 10 mg/ml transferrin, 5 mg/ml insulin, 2 mg/ml fetuin, 1% L-glutamine and 1% sodium pyruvate). pZMP6/zcub5 is diluted into 15-ml tubes to a total final volume of 640 μl with SF media. 35 μl of Lipofectamine™ is mixed with 605 μl of SF medium. The resulting mixture is added to the DNA mixture and allowed to incubate approximately 30 minutes at room temperature. Five ml of SF media is added to the DNA:Lipofectamine™ mixture. The cells are rinsed once with 5 ml of SF media, aspirated, and the DNA:Lipofectamine™ mixture is added. The cells are incubated at 37° C. for five hours, then 6.4 ml of Ham's F12/10% FBS, 1% PSN media is added to each plate. The plates are incubated at 37° C. overnight, and the DNA:Lipofectamine™ mixture is replaced with fresh 5% FBS/Ham's media the next day. On day 3 post-transfection, the cells are split into T-175 flasks in growth medium. On day 7 postransfection, the cells are stained with FITC-anti-CD8 monoclonal antibody (Pharmingen, San Diego, Calif.) followed by anti-FITC-conjugated magnetic beads (Miltenyi Biotec). The CD8-positive cells are separated using commercially available columns (mini-MACS columns; Miltenyi Biotec) according to the manufacturer's directions and put into DMEM/Ham's F12/5% FBS without nucleosides but with 50 nM methotrexate (selection medium).

Cells are plated for subcloning at a density of 0.5, 1 and 5 cells per well in 96-well dishes in selection medium and allowed to grow out for approximately two weeks. The wells are checked for evaporation of medium and brought back to 200 μl per well as necessary during this process. When a large percentage of the colonies in the plate are near confluency, 100 μl of medium is collected from each well for analysis by dot blot, and the cells are fed with fresh selection medium. The supernatant is applied to a nitrocellulose filter in a dot blot apparatus, and the filter is treated at 100° C. in a vacuum oven to denature the protein. The filter is incubated in 625 mM Tris-glycine, pH 9.1, 5 mM β-mercaptoethanol, at 65° C., 10 minutes, then in 2.5% non-fat dry milk in Western A Buffer (0.25% gelatin, 50 mM Tris-HCl pH 7.4, 150 mM NaCl, 5 mM EDTA, 0.05% Igepal CA-630) overnight at 4° C. on a rotating shaker. The filter is incubated with the antibody-HRP conjugate in 2.5% non-fat dry milk in Western A buffer for 1 hour at room temperature on a rotating shaker. The filter is then washed three times at room temperature in PBS plus 0.01% Tween 20, 15 minutes per wash. The filter is developed with chemiluminescence reagents (ECL™ direct labelling kit; Amersham Corp., Arlington Heights, Ill.) according to the manufacturer's directions and exposed to film (Hyperfilm ECL, Amersham Corp.) for approximately 5 minutes.

Positive clones are trypsinized from the 96-well dish and transferred to 6-well dishes in selection medium for scaleup and analysis by Western blot.

### Example 9

Full-length zcub5 protein is produced in BHK cells transfected with pZMP6/zcub5 (Example 7). BHK 570 cells (ATCC CRL-10314) are plated in 10-cm tissue culture dishes and allowed to grow to approximately 50 to 70% confluence overnight at 37° C., 5% CO₂, in DMEM/FBS media (DMEM, Gibco/BRL High Glucose; Life Technologies), 5% fetal bovine serum (Hyclone, Logan, Utah), 1 mM L-glutamine (JRH Biosciences, Lenexa, Kans.), 1 mM sodium pyruvate (Life Technologies). The cells are then transfected with pZMP6/zcub5 by liposome-mediated transfection (using Lipofectanine™; Life Technologies), in serum free (SF) media (DMEM supplemented with 10 mg/mil transferrin, 5 mg/ml insulin, 2 mg/ml fetuin, 1% L-glutamine, and 1% sodium pyruvate). The plasmid is diluted into 15-ml tubes to a total final volume of 640 μl with SF media. 35 μl of the lipid mixture is mixed with 605 μl of SF medium, and the resulting mixture is allowed to incubate approximately 30 minutes at room temperature. Five milliliters of SF media is then added to the DNA:lipid mixture. The cells are rinsed once with 5 ml of SF media, aspirated, and the DNA:lipid mixture is added. The cells are incubated at 37° C. for five hours, then 6.4 ml of DMEM/10% FBS, 1% PSN media is added to each plate. The plates are incubated at 37° C. overnight, and the DNA:lipid mixture is replaced with fresh 5% FBS/DMEM media the next day. On day 5 post-transfection, the cells are split into T-162 flasks in selection medium (DMEM +5% FBS, 1% L-Gln, 1% sodium pyruvate, 1 μM methotrexate). Approximately 10 days post-transfection, two 150-mm culture dishes of methotrexate-resistant colonies from each transfection are trypsinized, and the cells are pooled and plated into a T-162 flask and transferred to large-scale culture.

### Example 10

For construction of adenovirus vectors, the protein coding region of human zcub5 is amplified by PCR using primers that add PmeI and AscI restriction sites at the 5′ and 3′ termini respectively. Amplification is performed with a full-length zcub5 cDNA template in a PCR reaction as follows: incubation at 95° C. for 5 minutes; followed by 15 cycles at 95° C. for 1 min., 61° C. for 1 min., and 72° C. for 1.5 min.; followed by 72° C. for 7 min.; followed by a 4° C. soak. The reaction product is loaded onto a 1.2% low-melting-temperature agarose gel in TAE buffer (0.04 M Tris-acetate, 0.001 M EDTA). The zcub5 DNA is excised from the gel and purified using a commercially available kit comprising a silica gel mambrane spin column (QIAquick™ PCR Purification Kit and gel cleanup kit; Qiagen, Inc.) as per kit instructions. The zcub5 DNA is then digested with PmeI and AscI, phenol/chloroform extracted, EtOH precipitated, and rehydrated in 20 ml TE (Tris/EDTA pH 8). The resulting zcub5 fragment is then ligated into the PmeI-AscI sites of the transgenic vector pTG12-8 and transformed into E. coli DH10B™ competent cells by electroporation. Vector pTG 12-8 was derived from p2999B4 (Palniter et al., Mol. Cell Biol. 13:5266-5275, 1993) by insertion of a rat insulin II intron (ca. 200 bp) and polylinker (Fse I/Pme I/Asc I) into the Nru I site. The vector comprises a mouse metallothionein (MT-1) promoter (ca. 750 bp) and human growth hormone (hGH) untranslated region and polyadenylation signal (ca. 650 bp) flanked by 10 kb of MT-1 5′ flanking sequence and 7 kb of MT-1 3′ flanking sequence. The cDNA is inserted between the insulin II and hGH sequences. Clones containing zcub5 are identified by plasmid DNA miniprep followed by digestion with PmeI and AscI. A positive clone is sequenced to insure that there were no deletions or other anomalies in the construct.

DNA is prepared using a commercially available kit (Maxi Kit, Qiagen, Inc.), and the zcub5 cDNA is released from the pTG12-8 vector using PmeI and AscI enzymes. The cDNA is isolated on a 1% low melting temperature agarose gel and excised from the gel. The gel slice is melted at 70° C., and the DNA is extracted twice with an equal volume of Tris-buffered phenol, precipitated with EtOH, and resuspended in 10 μl H₂O.

The zcub5 cDNA is cloned into the EcoRV-AscI sites of a modified pAdTrack-CMV (He, T-C. et al., Proc. Natl. Acad. Sci. USA 95:2509-2514, 1998). This construct contains the green fluorescent protein (GFP) marker gene. The CMV promoter driving GFP expression is replaced with the SV40 promoter, and the SV40 polyadenylation signal is replaced with the human growth hormone polyadenylation signal. In addition, the native polylinker is replaced with FseI, EcoRV, and AscI sites. This modified form of pAdTrack-CMV is named pZyTrack. Ligation is performed using a commercially available DNA ligation and screening kit (Fast-Link™ kit; Epicentre Technologies, Madison, Wis.). Clones containing zcub5 are identified by digestion of mini prep DNA with FseI and AscI. In order to linearize the plasmid, approximately 5 μg of the resulting pZyTrack zcub5 plasmid is digested with PmeI. Approximately 1 μg of the linearized plasmid is cotransformed with 200 ng of supercoiled pAdEasy (He et al., ibid.) into E. coli BJ5183 cells (He et al., ibid.). The co-transformation is done using a Bio-Rad Gene Pulser at 2.5 kV, 200 ohms and 25 μFa. The entire co-transformation mixture is plated on 4 LB plates containing 25 μg/ml kanamycin. The smallest colonies are picked and expanded in LB/kanamycin, and recombinant adenovirus DNA is identified by standard DNA miniprep procedures. The recombinant adenovirus miniprep DNA is transformed into E. coli DH10B™ competent cells, and DNA is prepared using a Maxi Kit (Qiagen, Inc.) aaccording to kit instructions.

Approximately 5 μg of recombinant adenoviral DNA is digested with PacI enzyme (New England Biolabs) for 3 hours at 37° C. in a reaction volume of 100 μl containing 20-30U of PacI. The digested DNA is extracted twice with an equal volume of phenol/chloroform and precipitated with ethanol. The DNA pellet is resuspended in 10 μl distilled water. A T25 flask of QBI-293A cells (Quantum Biotechnologies, Inc. Montreal, Qc. Canada), inoculated the day before and grown to 60-70% confluence, is transfected with the PacI digested DNA. The PacI-digested DNA is diluted up to a total volume of 50 μl with sterile HBS (150 mM NaCl, 20 mM HEPES). In a separate tube, 20 μl of 1 mg/ml N-[1-(2,3-Dioleoyloxy)propyl]-N,N,N-trimethyl-ammonium salts (DOTAP) (Boehringer Mannheim, Indianapolis, Ind.) is diluted to a total volume of 100 μl with HBS. The DNA is added to the DOTAP, mixed gently by pipeting up and down, and left at room temperature for 15 minutes. The media is removed from the cells and washed with 5 ml serum-free minimum essential medium (MEM) alpha containing 1 mM sodium pyruvate, 0.1 mM MEM non-essential amino acids, and 25 mM HEPES buffer (reagents obtained from Life Technologies, Gaithersburg, Md.). 5 ml of serum-free MEM is added, and the cells are held at 37° C. The DNA/lipid mixture is added drop-wise to the flask of cells, mixed gently, and incubated at 37° C. for 4 hours. After 4 hours the media containing the DNA/lipid mixture is aspirated off and replaced with 5 ml complete MEM containing 5% fetal bovine serum. The transfected cells are monitored for GFP expression and formation of foci (viral plaques).

Seven days after transfection of 293A cells with the recombinant adenoviral DNA, cells expressing GFP start to form foci. The crude viral lysate is collected using a cell scraper to collect the cells. The lysate is transferred to a 50-ml conical tube. To release most of the virus particles from the cells, three freeze/thaw cycles are done in a dry ice/ethanol bath and a 37° waterbath.

The crude lysate is amplified (Primary (1°) amplification) to obtain a working stock of zcub5 rAdV lysate. Ten 10 cm plates of nearly confluent (80-90%) 293A cells are set up 20 hours previously, 200 ml of crude rAdV lysate is added to each 10-cm plate, and the cells are monitored for 48 to 72 hours for CPE (cytopathic effect) under the white light microscope and expression of GFP under the fluorescent microscope. When all the cells show CPE, this 1° stock lysate is collected and freeze/thaw cycles are performed as described above.

A secondary (2°) amplification of zcub5 rAdV is then performed. Twenty 15-cm tissue culture dishes of 293A cells are prepared so that the cells are 80-90% confluent. All but 20 ml of 5% MEM media is removed, and each dish is inoculated with 300-500 ml of the 1° amplified rAdv lysate. After 48 hours the cells are lysed from virus production, the lysate is collected into 250-ml polypropylene centrifuge bottles, and the rAdV is purified.

NP-40 detergent is added to a final concentration of 0.5% to the bottles of crude lysate in order to lyse all cells. Bottles are placed on a rotating platform for 10 minutes agitating as fast as possible without the bottles falling over. The debris is pelleted by centrifugation at 20,000× G for 15 minutes. The supernatant is transferred to 250-ml polycarbonate centrifuge bottles, and 0.5 volume of 20% PEG8000/2.5 M NaCl solution is added. The bottles are shaken overnight on ice. The bottles are centrifuged at 20,000× G for 15 minutes, and the supernatant is discarded into a bleach solution. Using a sterile cell scraper, the white, virus/PEG precipitate from 2 bottles is resuspended in 2.5 ml PBS. The resulting virus solution is placed in 2-ml microcentrifuge tubes and centrifuged at 14,000× G in the microcentrifuge for 10 minutes to remove any additional cell debris. The supernatant from the 2-ml microcentrifuge tubes is transferred into a 15-ml polypropylene snapcap tube and adjusted to a density of 1.34 g/ml with CsCl. The solution is transferred to 3.2-ml, polycarbonate, thick-walled centrifuge tubes and spun at 348,000× G for 3-4 hours at 25° C. The virus forms a white band. Using wide-bore pipette tips, the virus band is collected.

A commercially available ion-exchange columns (e.g., PD-10 columns prepacked with Sephadex® G-25M; Pharmacia Biotech, Piscataway, N.J.) is used to desalt the virus preparation. The column is equilibrated with 20 ml of PBS. The virus is loaded and allowed to run into the column. 5 ml of PBS is added to the column, and fractions of 8-10 drops are collected. The optical densities of 1:50 dilutions of each fraction are determined at 260 nm on a spectrophotometer. Peak fractions are pooled, and the optical density (OD) of a 1:25 dilution is determined. OD is converted to virus concentration using the formula: (OD at 260 nm)(25)(1.1×10¹²)=virions/ml.

To store the virus, glycerol is added to the purified virus to a final concentration of 15%, mixed gently but effectively, and stored in aliquots at −80° C.

A protocol developed by Quantum Biotechnologies, Inc. (Montreal, Canada) is followed to measure recombinant virus infectivity. Briefly, two 96-well tissue culture plates are seeded with 1×10⁴293A cells per well in MEM containing 2% fetal bovine serum for each recombinant virus to be assayed. After 24 hours 10-fold dilutions of each virus from 1×10⁻² to 1×10⁻¹⁴ are made in MEM containing 2% fetal bovine serum. 100 μl of each dilution is placed in each of 20 wells. After 5 days at 37° C., wells are read either positive or negative for CPE, and a value for plaque forming units/ml is calculated.

From the foregoing, it will be appreciated that, although specific embodiments of the invention have been described herein for purposes of illustration, various modifications may be made without deviating from the spirit and scope of the invention. Accordingly, the invention is not limited except as by the appended claims.

What is claimed is:

1. An isolated polypeptide comprising residues 41-150 of SEQ ID NO:2 or residues 32-141 of SEQ ID NO:4.

2. The isolated polypeptide of claim 1 which is not more than 1800 amino acid residues in length.

3. The isolated polypeptide of claim 2, wherein said residues 41-150 of SEQ ID NO:2 or residues 32-141 of SEQ ID NO:4 are operably linked to a second polypeptide selected from the group consisting of maltose binding protein, an immunoglobulin constant region, a polyhistidine tag, a peptide as shown in SEQ ID NO:7, and a peptide linker consisting of up to 25 amino acid residues.

4. The isolated polypeptide of claim 1, comprising residues 41-150 of SEQ ID NO:2.

5. The isolated polypeptide of claim 4, comprising a sequence of amino acid residues selected from the group consisting of:
residues 41-412 of SEQ ID NO:2; and
residues 41-452 of SEQ ID NO:2.
residues 35-150 of SEQ ID NO:2;
residues 35-412 of SEQ ID NO:2; and
residues 35-452 of SEQ ID NO:2.

6. The isolated polypeptide of claim 5 further comprising an immunoglobulin constant region domain and hinge region.

7. A dimerized polypeptide fusion comprising two polypeptide chains, each of said chains comprising residues 41 to 150 of SEQ ID NO:2 joined to an IgG constant region domain and hinge region.

8. The dimerized polypeptide fusion of claim 7, wherein each of said chains comprises residues 41 to 412 of SEQ ID NO:2 joined to an IgG constant region domain and hinge region.

9. An isolated polynucleotide encoding residues 41-150 of SEQ ID NO:2 or residues 32-141 of SEQ ID NO:4.

10. An expression vector comprising the following operably linked elements:
(a) a transcription promoter;
(b) a DNA segment encoding a polypeptide comprising a sequence of amino acid residues selected from the group consisting of:
residues 41-150 of SEQ ID NO:2;
residues 41-412 of SEQ ID NO:2;
residues 41-452 of SEQ ID NO:2;
residues 35-150 of SEQ ID NO:2;
residues 35-412 of SEQ ID NO:2;
residues 35-452 of SEQ ID NO:2;
residues 32-141 of SEQ ID NO:4;
residues 32-244 of SEQ ID NO:4;
residues 26-141 of SEQ ID NO:4; and
residues 26-244 of SEQ ID NO:4; and
(c) a transcription terminator.

11. The expression vector of claim 10 further comprising a secretory signal sequence operably linked to the DNA segment.

12. The expression vector of claim 11, wherein the secretory signal sequence encodes residues 1-34 of SEQ ID NO:2 or residues 1-25 of SEQ ID NO:4.

13. The expression vector of claim 10 wherein said polypeptide further comprises a maltose binding protein, an immunoglobulin constant region, a polyhistidine tag, a peptide as shown in SEQ ID NO:7, or a peptide linker consisting of up to 25 amino acid residues.

14. The expression vector of claim 13 wherein said polypeptide further comprises an immunoglobulin constant region domain and hinge region.

15. A cultured cell into which has been introduced the expression vector of claim 10, wherein said cell expresses said DNA segment.

16. A method of making a protein comprising:
culturing the cell of claim 15 under conditions whereby the DNA segment is expressed and the polypeptide is produced; and
recovering the polypeptide.

17. The method of claim 16 wherein the expression vector comprises a secretory signal sequence operably linked to the DNA segment, and wherein the polypeptide is secreted by the cell and recovered from a medium in which the cell is cultured.

18. A polypeptide produced by the method of claim 16.

19. An antibody that specifically binds to the polypeptide of claim 1.
